(19) United States
(12) Patent Application Publication
(10) Pub. No.: US 2024/0292877 A1
(43) Pub. Date: Sep. 5, 2024

(54) EDIBLE BIOREACTORS AND COMPOSITIONS THEREOF

(71) Applicant: Foodberry, Inc., Boston, MA (US)

(72) Inventors: Martin KOLEWE, Needham, MA (US); Christopher HANGO, Boston, MA (US); Kristen ENRIGHT, Bedford, MA (US); David EDWARDS, Boston, MA (US)

(21) Appl. No.: 18/573,119

(22) PCT Filed: Jun. 24, 2022
(86) PCT No.: PCT/US2022/034950

§ 371 Date: Dec. 21, 2023

Related U.S. Application Data
(60) Provisional application No. 63/215,417, filed on Jun. 26, 2021.

(51) Int. Cl. A23P 10/30; A23C 9/13; A23F 3/16; A23L 19/20

(52) U.S. Cl. CPC A23P 10/30; A23C 9/1307; A23F 3/163; A23F 3/166; A23L 19/20; A23C 2210/40

(57) ABSTRACT

The present disclosure relates to edible compositions, and more specifically, edible bioreactors comprising an edible membrane wherein the membrane is a bioreactor vessel, the membrane encapsulates a bioreactor vessel or a composition comprising active biological materials, or the membrane causes, affects, or interacts with a bioreaction; in addition to processes for making the edible bioreactors, and methods for producing edible products using the edible bioreactors.

### RELATED APPLICATIONS

This application claims the benefit of U.S. Provisional Application No. 63/215,417, filed on Jun. 26, 2021, the entire disclosure of which is incorporated herein by reference.

### BACKGROUND

Certain foods, supplements, and pharmaceuticals are the result of one or more bioreactions. A “bioreaction” is a biological active process or a chemical process involving organisms or biochemically active substances derived from such organisms. A “bioreactor” is a manufactured composition, device, apparatus, or system that supports a bioreaction, including for example, reactions in which living organisms, such as bacteria, or biochemically active substances derived from such organisms produce, synthesize, break down, or transform molecules. A bioreactor, for example, may include a vessel for a bioreaction or a body that supports a biologically active environment. A “bioreaction product” is any substance produced or resulting from a bioreaction, where a “bioreaction byproduct” generally means any secondary or undesired materials produced in addition to the bioreaction products.

Many foods are created through bioreactions, including fermentation of beer, yogurt, or cheese by yeast or bacteria. Today, manufacturing foods using fermentation often requires industrial processes that require expensive manufacturing equipment, including manufacturing lines with one or more vessels that may be employed during different stages.

To illustrate, a manufacturing system to produce yogurt may first require standardizing, adding or combining raw materials, homogenizing, heating/pasteurizing, cooling, and/or inoculating the raw materials, also known as upstream processes, to prepare them for a bioreaction. Upstream processing is often done in one or more steps in the production line with a mechanism to move the materials from one area to a different area in the production line (for example, from a first vessel to a second vessel). After the upstream steps, the contents often must be transferred again to one or more bioreactors for the bioreactions to occur. Finally, the bioreaction product is often processed after the bioreaction, i.e., downstream processing, which may require one or more steps which may occur in one or more vessels.

Bioreactors and related processes often require expensive and highly-specialized equipment. Bioreactions, particularly those used in pharmaceutical and food production, are often highly-sensitive to process conditions (e.g., temperature, pressure, pH, atmospheric composition, contamination, etc.) and thus must usually be sufficiently isolated from the surrounding environment and well-controlled. As a result, the costs required to produce manufactured materials through bioreactions can be substantial. There remains a need to streamline these processes and lower these costs.

As our understanding of biology and biomaterials deepens, there is a growing need for production of increasingly complex and personalized medicines. In particular, delivery vehicles must be developed for therapeutics comprising biologically active cells or cell-derived products (e.g., stein cells, antibodies, nucleic acids, probiotics, supplements, small molecule drugs, etc.). In this respect, edible bioreactors can effectively function as transport vessels for the delivery of such biologically active materials so that bioreactions can occur within the body, producing useful products. In many cases, delivery of biologically active cells in vivo can yield more robust, dynamic, and efficacious results as compared to simple administration of the molecules produced from these bioreactions in vitro. The need to deliver an edible bioreactor that may be consumed orally or otherwise delivered internally thus exists.

The exemplary edible bioreactors disclosed herein may be utilized to manufacture foods, supplements, or pharmaceuticals, lower the costs of producing or manufacturing foods or other substances with bioreaction processes, deliver medicinal bioreaction products, as well as for other uses described more fully herein.

### SUMMARY

In certain embodiments, an edible composition, particularly an edible bioreactor, comprises a core (e.g., an edible core) and an edible membrane encapsulating the core, wherein the membrane supports a bioreaction in the core.

In certain embodiments, an edible bioreactor comprises a core and a membrane encapsulating the core wherein the membrane supports a bioreaction in the core and wherein the membrane comprises (1) at least one edible polymer and edible particles or (2) a plurality of edible polymers.

In certain embodiments of the edible bioreactor, an edible bioreactor comprises a core, a first edible membrane encapsulating the core, and an edible second membrane encapsulating the first edible membrane wherein the first edible membrane inoculates the core and the second edible membrane supports a bioreaction.

In certain embodiments of the edible bioreactor, an edible bioreactor comprises a core and a membrane supporting the core wherein the membrane is a bioreactor vessel.

In certain embodiments of the edible bioreactor, an edible bioreactor comprises a core and a membrane wherein the membrane supports a bioreaction, upstream processes of the bioreaction, downstream processes of the bioreaction, or a combination thereof.

In certain embodiments of the edible bioreactor, an edible bioreactor comprises a core and an edible membrane encapsulating the core wherein the core is inoculated while encapsulated by the edible membrane.

In certain embodiments of the edible bioreactor, an edible bioreactor comprises a core, a first edible membrane encapsulating the core, and a second edible membrane encapsulating the first edible membrane wherein the core inoculates the first edible membrane and the second edible membrane supports a bioreaction.

In certain embodiments, an edible bioreactor comprising a core and an edible membrane encapsulating the core wherein the edible membrane is selectively permeable.

In certain embodiments, the core comprises a substrate which has not been subjected to a bioreaction prior to encapsulating in the edible membrane. For example, the bioreactor may comprise a substrate and an active culture, wherein the substrate is substantively reacted (e.g., fermented) only after encapsulating in the edible bioreactor. In certain embodiments, the core does not comprise a component which has been previously fermented. In certain embodiments, less than 50% by weight (e.g., less than 40 wt %, less than 35 wt %, less than 30 wt,%, less than 25 wt %, less than 20 wt %, less than 15 wt %, less than 10 wt %, less than 8 wt %, less than 6 wt %, less than 5 wt %, less than 4 wt %, less than 3 wt %, less than 2 wt %, or less than 1 wt %), of the components of the core have been previously fermented, such as an edible bioreactor that comprises a small quantity of yogurt containing active cultures).

In certain embodiments, the edible bioreactor supports a bioreaction (e.g. in the core) which is typically carried out at room temperature. For example, the edible bioreactors disclosed herein can support a fermentation by a mesophilic bacterial culture, such as a bacterial culture capable of fermenting milk into yogurt or cheese.

In certain embodiments, the edible bioreactor comprises an edible membrane that can support a bioreaction, e.g., in the core. For example, the edible membrane may withstand the conditions of the bioreaction, e.g., be stable to a temperature, pH, product, or side-product of the bioreaction, or the active culture or a product or component thereof (e.g., enzyme). In certain embodiments, the bioreaction supported by an edible membrane or edible bioreactor may not involve conditions known to rapidly and/or substantially denature, deform, degrade, or destroy the edible membrane (e.g., an alginate, such as sodium alginate), such as temperatures greater than, e.g., 50° C., 75° C., 100° C., 200° C., 300° C., or higher), or a pH-J lower than 3, e.g., lower than 2, lower than 1, or a pH greater than 9, e.g., greater than 10, or greater than 11.

In certain embodiments, an edible membrane of the edible bioreactor is selectively permeable. For example, the edible membrane may be substantially permeable to gas, such as a gas produced during the bioreaction (e.g., carbon dioxide), but substantially impermeable to a liquid and/or solid. This property of the edible bioreactor is advantageous, as it permits the release of a gaseous product or by-product from the edible bioreactor formed during a bioreaction, thereby preventing undesired expansion or deformation of the edible bioreactor and/or potential rupture of the edible membrane, and also avoids loss of liquid or solid components which may include an edible product and/or substrate.

### BRIEF DESCRIPTION OF THE DRAWINGS

FIG. 1 is a diagram of an exemplary embodiment of an edible bioreactor.

FIG. 2 is an illustration of an exemplary bioreactor with a temporary or conditional barrier between a culture and a substrate.

FIG. 3 is a diagram of an exemplary edible bioreactor with two barriers.

FIG. 4 is a diagram of exemplary edible bioreactor with a core that contains multiple core units, each core encapsulated by one or more edible membrane units.

FIG. 5 is an illustration of an exemplary edible bioreactor with a core containing multiple types of core units each encapsulated by one or more edible membrane units.

### DETAILED DESCRIPTION

Edible bioreactors can contain and protect ingestible/edible substances, such as food or beverages, within edible or biodegradable membranes (matrix or matrices) and/or shells, and can support a bioreaction, e.g., within the core of the bioreactor. The edible membranes/shells of edible bioreactors can be formed from various substances allowing different compositions to be transported and consumed. As used herein, the terms “membrane(s),” “matrix” or “matrices,” and “shell(s)” may refer to similar or different materials or kinds of materials, depending on the type of object, how many barrier layers of any sort it may have, or the properties and contents of any such barrier layers. Thus, for some embodiments, the terms can be used interchangeably. In certain embodiments, membranes and/or membranes and shells are edible, providing nutritious benefits as well as reducing concerns about littering and waste. Embodiments of the edible bioreactor described herein can have, e.g., varying shell or membrane thickness, one or more of a variety of chemical constituents, varying numbers of membranes, varying permeabilities, various consumable payloads, various shapes, and are constructed from various shell/membrane properties to provide a variety of flavors and textures and membrane characteristics and to support a variety of bioreactions. Embodiments of the edible bioreactors can be made at large scale, using, for example, injection techniques, spray and spray drying techniques, fluidized-bed, and other technologies. See, for example, U.S. Pat. No. 11,172,690 and PCT application WO 2011/103594, hereby incorporated in their entirety.

The core, as used herein, may comprise (e.g., consist of) edible materials that are generally solid, semi-solid or liquid in form, and may be capable of providing nutrition when consumed, and are typically provided in a form suitable for ingestion. The core may be referred to herein as the edible core. Edible materials can be derived from many sources including plants and animals, particularly those generated by agriculture, or from artificial production methods including chemical synthesis. Edible refers to any substance that can provide for an organism's (e.g., a human or other mammal) nutritional needs or sensory desires, typically when consumed orally, and is usually non-toxic when properly consumed. Biodegradable refers to capable of being decomposed by actions of biological agents such as microorganisms, or by non-biological effects such as environmental exposure. Liquid refers to having a consistency like that of water or oil, that is to say, flowing freely but of constant volume. Solid refers to being characterized by structural rigidity and resistance to changes of shape and volume. Semi-solid refers to having a rigidity intermediate between a solid and a liquid. Viscosity refers to a fluid's resistance to flow, wherein gel-like liquids have higher viscosity—for example, ketchup is more viscous than water. Foam refers to a mass of small bubbles formed on or in a substrate, typically a liquid, but also includes ice cream, frozen yogurts, and gelato. Frozen refers to a phase change in which a liquid is turned into a solid when its temperature is lowered beyond its freezing point. In some embodiments, the food material may be liquid, partially liquid, viscous, partially or fully solid, or contain several states of matter having different degrees of liquidity or solidness.

Ingestible substances include those that are edible or potable such as, for example, juice, chocolate, yogurt, beer, kombucha, sauerkraut, kefir, milk, cheese, various medicines, and various other solids, liquids, slurries, emulsions, foams, etc. For example, foods, particularly fruits and vegetables, such as berries, plants, and beans, are provided in various states of matter: liquid, semi-solid, solid, and frozen. They can be mixed with each other and optionally one or more nutrients and additives in varying proportions can be added to the mixture to produce a large variety of novel food objects. Their texture and consistency can be manipulated by physical, chemical, or biochemical means. Ingestible substances may be participants in or products of a bioreaction.

Membranes and shells of edible bioreactors may be made by using any one of many edible and/or biodegradable polymers. Alginate (alginic acid) is an example of a polymer that can be used in forming a membrane of an edible bioreactor disclosed herein. Alginate is an anionic, polymeric polysaccharide, widely present in the cell walls of brown algae. It is a copolymer of the structure -(M)ₘ-(G)ₙ- with segments composed of mannuronate M (mannuronic acid) and guluronate G (guluronic acid) monomeric subunits. When used in an edible membrane disclosed herein, the values of m and n, the ratio m/n, and the space distribution between M and G (i.e., presence of consecutive G-subunits and M-subunits, or randomly organized subunits) may all play key roles in the chemical and physical properties of the edible membrane.

Alginates have been applied to pharmaceutical preparations, impression-making materials (e.g., in dentistry and in prosthetics manufacturing), and in the food industry. Sodium alginates also have found application in restaurants, e.g., to create spheres of liquid surrounded by a thin jelly membrane. Modern chefs such as Ferran Adria have used sodium alginates to create “melon caviar,” “false fish eggs,” etc., by adding sodium alginates into a liquid (e.g., melon juice), then dropping the preparation in a calcium bath (calcium lactate or calcium chloride). Beyond their biocompatibility to human use, polymers such as alginate have the capacity to easily form a gel. To induce rapid gelation by electrostatic cross-linking, the naturally present Na⁺ ions are removed and replaced by divalent cations (e.g., Ca²⁺ or another multi-valent cation such as Mg²⁺).

The approach disclosed herein involves forming encapsulated vessels or edible membranes that can use various particles, particulates, and polymers, in combination or separately, to create desired properties of strength, stability, permeability, edibility, and biodegradability for the support of a bioreaction that, in certain embodiments, is part of an edible bioreactor, that can be easily moved and consumed. As used herein, the terms particle(s) and particulate(s) are used interchangeably. In some embodiments, a consumable, a bioactive core, or a core capable of undergoing or participating in a bioreaction, is encased in a polysaccharide membrane, for example, an alginate membrane. Methods for encasing a consumable edible product are found in U.S. Pat. No. 11,172,690; U.S. patent application Ser. Nos. 14/374,069, 14/908,789, 61/591,054, 61/601,852, 61/591,262, 61/591,233, 61/591,225, 61/647,721, 61/713,138, 61/713,100, 61/601,866, and 61/713,063; and PCT Application Nos. WO 2013/113027, WO 2014/151326, WO 2015/017625, WO2014/028654; each of which are herein incorporated in their entireties.

In some embodiments, ingestible particles are embedded in a membrane (e.g., a membrane of an edible bioreactor disclosed herein), which may improve the physical, chemical and/or physicochemical characteristics of the membrane, and/or to improve the membrane's ability to support a bioreaction (e.g., within the core of the bioreactor), and/or impart the ability of the membrane to optimize, influence, or control the bioreaction that it supports. In addition, in certain embodiments, the ingestible particles impart a flavor, for example chocolate or various fruit flavors, wherein such particles may be embedded before, during, or after the bioreaction in the edible membrane.

It is possible to vary membrane component concentrations (for example, decreasing the membrane polymer concentration and increasing the membrane particulate concentration) by using particles that are charged, such as particles that possess the same charge state as other membrane polymers or particulates, which may improve the performance for bioreactions. In certain embodiments of, for example, an alginate-based membrane, when particles carry the opposite charge state as alginate polymers or particulates, one can minimize or eliminate the need for a calcium solution or another multivalent ion by using particles to bind with alginates or another charged polymer. For non-alginate-based systems, combinations of or homogenous particles can be used to encapsulate the edible material, or can be used in combination with polymers at lower weight %-by-mass than the particles (for example, less than 80%, less than 70%, less than 60%, less than 50%, less than 40%, less than 30%, less than 20%, less than 10% polymer). In certain embodiments, a thinner membrane can be sufficient to encapsulate a larger quantity of ingestible material, which may have further advantages of taste and texture. Particles contemplated herein include large food particles, for example with average diameters greater than 1 millimeter (linseeds, sesame seeds, poppy seeds, chia seeds, chopped or pulverized foods including fruits, fruit skins, vegetables, etc.), small grains, and pulverized seeds, nuts, etc. In some embodiments, compositions use particulates with an average diameter less than about 1 millimeter.

The term “about” is used herein to mean within the typical ranges of tolerances in the art. For example, “about” can be understood as about 2 standard deviations from the mean. In some embodiments, “about” means ±10%. In “some” embodiments, about means ±5%. When “about” is present before a series of numbers or a range, it is understood that “about” can modify each of the numbers in the series or range.

In certain embodiments, particulates used for the membrane(s) can advantageously affect the membrane strength, diffusion permeability (including, for example, achieving selective permeability, e.g., to gas), and stability to optimize, influence, or control a bioreaction in a core. Certain variables when considering particulates as components for membranes include: (1) the particle charge or net charge of a heterogenous or homogenous particulate mix, (2) the specific combinations of particulates for a heterogenous mix, (3) the hygroscopic or hydrophilic nature of the particulates, (4) the solubility of particulates in a liquid polymer, (5) the aqueous solubility of the particles, (6) the particle solubility in polar, non-polar, or amphipathic solvents, (7) the particle size, (8) the heterogeneity of particle size, (9) the heterogeneity of particle sizes in a heterogenous or homogenous mix of particles, (10) the shape of particulates in a heterogenous or homogenous mix of particles, and (11) the chemical and physical nature of the edible or potable substance to be encased in the membrane when interacting with the particulates. In addition, for membranes that support a bioreactor, other variables when considering particulates as components for membranes include: (1) the products and byproducts of a bioreaction, (2) the permeability of the membrane, (3) the conditions suitable to support a bioreaction, (4) the bioactive material, (5) the substrate or medium, (6) the elasticity of the membrane, and (7) the changes in the core due to the bioreaction, including changes relating to size, solubility, shape, composition, pH, and changes in states of matter.

In some embodiments, the particles are neutrally charged. In some embodiments, the particulates have various charge states, and can have an opposite charge as the membrane polymer or other membrane constituents. The overall charge state of the membrane polymer or other membrane constituents can influence the choice of particulates, as particles oppositely charged to the charge state of the membrane polymer or particle matrix are likely incorporated into the membrane matrix and preferentially bonded. Oppositely charged particles could contribute to the formation of salt bridges within the membrane matrix and/or membrane polymeric subunit architecture.

In certain embodiments, polysaccharide polymers are used as the membrane polymer. Polysaccharide polymer based membranes can be porous, and porosity may be determined by the chemical content and 2- and 3-dimensional geometry of the polymeric structure of the membrane, for example the structure of the polysaccharide chain. Therefore, particulates that can be used in the bioreactors disclosed herein can be appropriately accommodated by the pore structure of the membrane, whether as particles that can be intercalated between polymeric chains and/or embedded into the pores to act as a plug based on a particulate size and shape, electrostatically bind to create salt bridges, enhance Van der Waals interactions that can contribute to overall membrane stability, etc. As described herein various physical and chemical characteristics of the particulates can be matched to the membrane structure and chemistry to achieve a desired effect, for example increased impermeability, elasticity, membrane strength-to-weight ratio, color, syneresis, etc., including the desired effect to allow the membrane to support a bioreaction, and to optimize, influence, or control the supported bioreaction.

In some embodiments, the particulates used for the membrane are sized (e.g., having an average diameter) at about 0.01 microns, at about 0.1 microns, at about 0.1 to 1.0 microns, at about 0.1 to 10 microns, at about 0.1 to 100 microns, at about 0.01 to about 1 millimeter, or to about 3 millimeters, or at about 0.1 to about 1 millimeter, or to about 3 millimeters. The size of the particulates may be important for embedment characteristics into the porous structure of the membrane.

The porosity of membranes also can be determined in part by the ratios of the subunits and or the particulates that assemble to form the membrane. For example, alginate-based membranes are composed of mannuronic acid and guluronic acid subunits. In general, for alginates, increasing the number of guluronic acid subunits relative to the number of mannuronic acid subunits will contribute to a loss of mobility of the membrane polymers, resulting in a stiffer and more stable membrane. However, the stability may also be offset by increased porosity of the membrane. The overall concentration of polymer used when in solution (prior to forming a membrane) may also contribute to porosity of the membrane formed. All else being equal, increasing the concentration (or the density) of a polymer can decrease the porosity of the final membrane. However, other considerations such as consumer preference or gustatory experience when ingesting the membrane may also be considered for determining the range of desirable polymer concentrations. Therefore, ratios of polymeric building blocks and/or particulates of a membrane may be considered for determining membrane porosity with respect to particulate embedment, solution diffusion, and membrane permeability, and how these characteristics are related to each other and how they support, optimize, influence, or support a bioreaction supported by the membrane.

In certain embodiments, the molecular weight of the membrane polymer is between about 2,000 Daltons and about 2,000,000 Daltons or larger. In some embodiments, the polysaccharide polymer present in solution is between about 0.1% by weight and about 5% by weight, between about 0.1% and 10%, by weight, or greater.

In certain embodiments, not all of the particulates are incorporated into the membrane. Instead, in some embodiments, a layer of particulates remains unincorporated, and form a layer next to a membrane or between two or more membrane layers. The additional particulate layer can contribute to, for example, permeability, elasticity, strength, durability, syneresis, hygroscopy, hydrophobicity, etc., of the membrane, or changes across and within membrane layers. Thus, the chemical nature of the particulates, for example if a hydrophobic particulate is used, can contribute to impeding the diffusion of liquid across an inner layer to an outer layer surface boundary. In some embodiments, particulates can be layered so that the particulate layer has multiple effects, for example an inner impermeability layer, a middle flavor/texture/payload (e.g., a pharmaceutical or supplement) layer, and an outer strength improving layer. In some embodiments, the particulates can be layered so that one or more particulate layers is bioactive or causes or takes part in a bioreaction.

In some embodiments, the particulate used may serve as a flavoring agent, a sweetener, a bittering agent, or to impart a salty flavor. Various foods and flavorings in powdered or extract form are contemplated, including fruits, vegetables, herbs and spices, and various food salts (onion salt, garlic salt, sea salt, etc.). Some embodiments use any of a variety of herbal extracts, energy supplements, dietary supplements, pharmaceuticals, over-the-counter drugs, sleep aids, appetite suppressants, weight gain agents, antioxidants, nutraceuticals, confections, and the like. As used herein, over-the-counter drugs refers to pharmaceutical compounds and compositions that had required a prescription but have been released from such prescription requirement for purchase and consumption. In other embodiments, the particulate may be a bioreactor.

In some embodiments, the core can be coated in a plurality of membranes. In certain embodiments, the membrane layers are distinct and melded. In other embodiments, the membrane layers are separate and distinct from other membrane layers. In certain embodiments, the same polymer, particulate, or combination of polymer(s) and/or particulate(s) is used for each of the multi-membrane coatings as described herein. In certain embodiments, different polymers, particulates, or combinations of polymer(s) and/or particulate(s) are used for each membrane in a multi-membrane layer. In some embodiments, a multilayered outer membrane has the same polymer, particulate, or combination of polymer(s) and/or particulate(s) in each of the outer layers, but the membrane components are different than those used in, for example, the inner membrane or other inner membrane layers. In certain embodiments, the plurality of membranes causes or takes part in a bioreaction, including for example, a membrane that inoculates a core to cause a bioreaction.

To accomplish the use of the same membrane components in a multi-membrane layered system while keeping the layers separate and distinct, in some embodiments, the inner membrane is first constructed, with or without additional particulates and/or polymers incorporated into the inner membrane. The membrane coated substance can then be layered with one or more additional polymer/particulate layers of homogenous or heterogenous polymers/particulates, and then the particulate layer can be coated again with another membrane. The process may be repeated as many times as desired to construct a multilayered product. In some embodiments, one or more layers contribute to, partake in, or cause a bioreaction and one or more layers support the bioreaction.

Various membrane polymers are contemplated for use in the membrane forming layers. Considerations for choice of membrane polymers may include inherent physicochemical characteristics (charge states, functional groups, kinetic reaction rates of polymerization, ion complex formation and cross-linking, etc.), texture, polymerization characteristics, reactivity to chemical interactions, reactions, and/or conditions such as pH, ionic strength, specific ions and ratios of ions during polymerization, presence of complexing agents (e.g., phosphates, citrate, ethylenediaminetetraacetic acid (EDTA), acids, glucono-delta-lactone (GDL), etc), shielding susceptibility of electrostatic character of polymer and polymeric strands, and cost effectiveness, e.g., if used for commercial production. Polysaccharide polymers contemplated herein include, but are not limited to, shellac, various fibers and hydrocolloids such as alginate, an agar, a starch, a gelatin, carrageenan, xanthan gum, gellan gum, galactomannan, gum arabic, a pectin, a milk protein, a cellulosic, gum tragacanth and karaya, xyloglucan, curdlan, a cereal pf-glucan, soluble soybean polysaccharide, a bacterial cellulose, a microcrystalline cellulose, chitosan, inulin, an emulsifying polymer, konjac mannan/konjac glucomannan, a seed gum, and pullulan. Combinations of these polysaccharides are also contemplated herein. The membrane polymers may also be selected to allow the membrane to support, influence, or optimize a bioreaction.

Other membrane compounds considered for use as structure forming compounds to modify or be used in combination with a polymer-based membrane (for example, a membrane consisting of a polysaccharide) include bagasse, tapioca, chitosan, polylactic acid, processed seaweed, chocolate, starch, gum arabic, cellulose based fibers, natural and synthetic amino acids and polymers thereof, proteins, and sugars/sugar derivatives. Combinations of these compounds and compositions are also contemplated herein.

A multi-layered and/or multi-component membrane for edible bioreactors can have several advantages: increased longevity or freshness of the edible or potable substance; limited diffusion of aqueous components of membrane polymers or edible and potable substances; decreased water activity of the potable or edible payload; wider spectrum of taste sensation and experience by a consumer when powders of different flavors and mouthfeel sensations are used, for example, between layers in a multilayered composition, taste improvement of a pharmaceutical or over-the-counter drug(s) if used as the particulate, etc. Incorporation of particulates into the outermost membrane can modify membrane performance, for example, the prevention of the outer membrane from polymerizing and or mechanically bonding with the inner or proximate membrane layer. Unincorporated particulates also likely form a physical barrier between membranes so that a chemical or mechanical bonding between membranes does not occur. Electrostatic repulsion/attraction, hydrophobicity, and/or hydrophilicity of particulates and other solvent/solute interactions between particulates and membrane polymer components may also contribute to preventing an interaction between a polymerized layer and a non-polymerized membrane component.

In some embodiments of a multilayered membrane, the proximately located membrane layers are made using the same polymer and the same particulates. In some embodiments, the proximately located membrane layers are made using different polymers and the same particulates to form the multiple membrane layers. In some embodiments, the proximately located membrane layers are made using the same polymers and different particulates to form the multiple membrane layers. In some embodiments, the proximately located membranes layers are made using different polymers and different particulates to form the multiple membrane layers. In some embodiments, different membranes are chosen wherein there is no inherent chemical or mechanical bonding between the membrane layers, thereby requiring no addition of particulates to the outer surface of the innermost membrane.

In some embodiments, membrane components, for example polysaccharides or proteins, are chemically modified with methods and compositions well known in the art. Modifications are important for altering functional groups of the membrane components which, in turn, can alter polymerization characteristics, chemical characteristics, physicochemical characteristics, bonding propensities, electrostatics, hydrophobicity or hydrophilicity changes, diffusion propensity and resistance to diffusion, elasticity, stability, etc., in the final polymerized membrane. Modifications include, but are not limited to, carbamoylation, graft polymerization, etherification, esterification, reduction, oxidation, amination, halogenation, polymerization and degradation, complex formation with metals and salts, etc. See, for example, Chemical and Functional Properties of Food Saccharides (ISBN 978-0-8493-1486-5).

In some embodiments, various ions are employed for use in the polymerized membrane and related chemical processes. In, for example, the alginate polysaccharide membrane, ions can be used to form cross-linkages between and among individual polymer strands. Various ion/counter ion salt complexes are contemplated for use herein, including, but not limited to, divalent cations such as calcium, magnesium, manganese, iron, zinc; trivalent cations including, but not limited to, manganese and iron; and salts thereof including, but not limited to, calcium lactate and calcium chloride.

In some embodiments, it is contemplated herein that micelles are formed within membranes and between membrane layers and/or between the inner membrane and the edible or potable substance. Micelles can alter the taste experience or mouth feel for the final encased product. Additionally, micelles engineered into the final membrane coated product may contain other ingestibles including sweeteners, flavors (fruits, herbs and spices, etc.), herbal extracts, energy supplements, dietary supplements, pharmaceuticals, over-the-counter drugs, sleep aids, appetite suppressants, weight gain agents, antioxidants, nutraceuticals, confections, and the like.

Certain embodiments of natural and artificial flavors contemplated for particulates include, but are not limited to, stevia rebaudioside A, glycyrrhizin, thaumatin, sorbitol, erythritol, mannitol, monk fruit, pentadin, xylitol, brazen, sugar, dextrose, crystalline fructose, maltodextrin, trehalose, molasses, aspartame, aspartame acesulfame salt, neotame, acesulfame, saccharin, sucralose, neohesperidin dihydrochalcone, sodium, saccharin, cyclamates, alitame, and dulcin.

Flavoring compounds contemplated for use in the membrane may be used to give the formulation payload a taste preferred by the end user or increase or enhance particular flavors or the perception of flavors. Flavor choices can include any fruit or vegetable flavor, or any artificial flavor, to elicit a desired taste perception (sweetness, sourness, bitterness, saltiness and/or umami, and associated food or flavoring, e.g., mint taste), as well as herbal or plant flavors that can otherwise be considered non-food (e.g., cinnamon), such as coffee, chocolate, and other confectionary flavors. Other flavor compounds considered as a novelty flavoring include, for example, beer and other alcoholic beverages, hemp, vomitus, and novel combinations of flavors (e.g., beer flavoring with caffeine).

Generally, dietary supplements may be considered as vitamins and/or minerals taken in addition to naturally obtained vitamins/minerals in food. Dietary supplements can be taken (1) to enhance the physical well-being or state of health of the end user, (2) as a health related supplement, or (3) as supplements required for enhancing deficient vitamin/mineral states in the end user. Dietary supplements can also add to a higher quality or perceived quality of the health state of the end user.

In certain embodiments, dietary supplements contemplated for use as membrane particles include, but are not limited to, Ascorbic Acid (Vitamin C), B Vitamins, Biotin, Fat Soluble Vitamins, Folic Acid, Hydroxycitric Acid (HCA), Inositol, pyruvate, Mineral Ascorbates, Mixed Tocopherols, Niacin (Vitamin B3), Orotic Acid, Para-Aminobenzoic Acid (PABA), Pantothenates, Pantothenic Acid (Vitamin B5), Pyridoxine Hydrochloride (Vitamin B6), Riboflavin (Vitamin B2), Synthetic Vitamins, Thiamine (Vitamin B1), Tocotrienols, Vitamin A, Vitamin D, Vitamin E, Vitamin F, Vitamin K, Vitamin Oils, Vitamin Premixes, Vitamin-Mineral Premixes, Water Soluble Vitamins, arsenic, boron, calcium, chloride, chromium, cobalt, copper, fluorine, iodine, iron, magnesium, manganese, molybdenum, nickel, phosphorous, potassium, selenium, silicon, sodium, strontium, sulfur, vanadium and zinc.

Energy supplements are designed to boost mental or physical activity. Various embodiments of ingestible energy supplements contemplated for use in membrane formulations include, but are not limited to, American ginseng, Red ginseng, Siberian ginseng, maca, rhodiola, ginger, guarana, turmeric, acetyl-L-carnitine, L-carnitine, creaune, taurine, L-phenylalanine, L-arginine, tyrosine, acetyl-tyrosine, N-acetyl L-tyrosine, Ginkgo biloba, yerba-mate, kola nut, gotu kola, maitake, Cordyceps sinensis, guarana, acai-berry, L-theanine, caffeine, quercetin, synephrine, green tea extract, theophylline, epigallocatechin gallate (EGCG), capsaicin, bee pollen, alpha-lipoic acid, and 1,3-dimethylamylamine (geranium), D-ribose, Fo-Ti, cha de bugre extract, and St. John's wort.

Oral health compounds can contribute to decreasing unwanted bacterial flora and/or covering up unwanted odors and/or flavors. Control of the unwanted flora can decrease incidence of tooth decay, halitosis, and potentially contributes to long-term health benefits including reducing incidence of heart disease.

In certain embodiments, oral health compounds for use as membrane particles include, but are not limited to, fluoride, vitamin C, vitamin B, zinc, menthol, thymol, eucalyptus, sodium bicarbonate, vitamin K, chlorhexidine, and xylitol.

Weight loss compounds are commonly divided into groups categorized as appetite suppressants, acting to manipulate hormonal and chemical processes in the body that otherwise increase hunger and/or the sense of feeling satiated (e.g., anorectics such as epinephrine and norepinephrine/noradrenaline), fat or cholesterol uptake inhibitors (such as green tea extract), gastrointestinal fillers, and thermogenetic compounds which boost a normal metabolic rate of the individual and result in metabolism of fat stores, all of which are contemplated for use in the present disclosure. Weight loss compounds can be synthetic or natural or a bioreaction product.

In certain embodiments, weight loss compositions contemplated herein as particles for the membrane include, but are not limited to, hoodia, chitosan, chromium picolinate, conjugated linoleic acid, glucomannan, green tea extract, guar gum, guarana, guggul, senna, ephedra, bitter orange, fucoxanthin, white bean extract, vitamin D, human chorionic gonadotropin, resveratrol, capsaicin, chia, hoodia, L-carnitine, raspberry ketones, banana leaf, red clover, ginger, almonds, acai berry, flax seeds, leucine, and lipodrene.

Sleep-aid compounds can assist in slowing the metabolic resting rate of an individual to allow one to relax and gain more restful or longer sleep periods. In certain embodiments, sleep aid compositions contemplated herein for use as membrane particles include, but are not limited to melatonin, 5-hydroxytryptophan, 5-hydroxytrypatmine, diphenhydramine, doxylamine, benzodiazepine, kava, serenite, chamomile, phenibut, catnip herb, chamomile, glycine, hops, L-theanine, L-tryptophan, glycine, GABA, and valerian.

Various over-the-counter and prescription based (pharmaceutical) drugs are contemplated for easier ingestion, and in some instances a more pleasant taste, as would be experienced by the user.

Over-the-counter and/or prescription (pharmaceutical) drugs may be included in an edible bioreactor disclosed herein, and/or may be the product of a bioreaction disclosed herein. In certain embodiments, over-the-counter (OTC) and prescription (pharmaceutical) drugs contemplated for use (e.g., as a membrane particle, component, and/or product of a bioreaction) include, but are not limited to, amikacin, gentamicin, kanamycin, neomycin, netilmicin, tobramycin, paromomycin, geldanamycin, herbimycin, loracarbef, ertapenem, doripenem, imipenem/cilastatin, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, teicoplanin, vancomycin, telavancin, clindamycin, lincomycin, daptomycin, azithromycin, clarithromycin, dirithromycin, erythromycin, roxithromycin, troleandomycin, telithromycin, spectinomycin, aztreonam, furazolidone, nitrofurantoin, amoxicillin, ampicillin, azlocillin, carbenicillin, cloxacillin, dicloxacillin, flucloxacillin, mezlocillin, methicillin, nafcillin, oxacillin, penicillin, piperacillin, temocillin, ticarcillin, ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfonamidochrysoiodine, sulfacetamide, sulfadiazine, silver, sulfadiazine, sulfamethizole, sulfamethoxazole, sulfanilamide, sulfasalazine, sulfisoxazole, trimethoprim, trimethoprim-sulfamethoxazole, demeclocycline, doxycycline, minocycline, oxytetracycline, tetracycline, clofazimine, dapsone, capreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, linezolid, metronidazole, mupirocin, platensimycin, quinupristin/dalfopristin, rifaximin, thiamphenicol, tigecycline, tinidazole, Fluoxetine, sertraline, paroxetine, fluvoxamine, citalopram, escitalopram, mirtazapine, triazolam, quazepam, estazolam, temazepam, zolpidem eszopiclone zaleplon, Trazodone, citalopram, escitalopram, desvenlafaxine, duloxetine, milnacipran, venlafaxine, tramadol, sibutramine, etoperidone, lubazodone, nefazodone, trazodone, reboxetine, viloxazine, atomoxetine, bupropion, dexmethylphenidate, methylphenidate, amphetamine, dextroamphetamine, dextromethamphetamine, lisdexamfetamine, amitriptyline, butriptyline, clomipramine, desipramine, dosulepin, doxepin, imipramine, iprindole, lofepramine, melitracen, nortriptyline, opipramol, protriptyline, trimipramine, amoxapine, maprotiline, mianserin, mirtazapine, isocarboxazid, moclobemide, phenelzine, selegiline, tranylcypromine, pirlindone, buspirone, tandospirone, aripiprazole, vilazodone, quetiapine, agomelatine, nefazodone, quetiapine, asenapine, carbamazepine, lithium, olanzapine, valproic acid, alprazolam, lorazepam, chlordiazepoxide, clonazepam, etizolam, tofisopamn, Azelastine, cetirizine, clemastine, desloratadine, dimenhydrinate, doxylamine, fexofenadine, loratadine (Claritin), ketorolac tromethamine, pemirolast potassium, ketotifen, neodocromil sodium, loteprednol etabonate, ipratropium bromide, beclomethasone, dexamethasone, epinastine, fluticasone, oxymetazoline, triamcinolone, cromolyn sodium, flunisolide, mometasone, ciclesonide, carbinoxamine maleate, olopatadine, budesonide, montelukast, epinephrine, fluticasone furoate and levocetirizine, Celecoxib (Celebrex), etodolac (iodine), meloxicam (Mobic), rofecoxib (Vioxx), valdecoxib (Bextra), ibuprofen, naproxen, diclofenac, flurbiprofen, indomethacin, ketoprofen, ketorolac, nabumetone, oxaprozin, piroxicam, sulindac, Aspirin, Acetaminophen, Pseudoephedrine HCl, Dextromethorphan, Chlorpheniramine Maleate, Pseudoephedrine HCl, Xylometazoline, Benzododecinium, Butamirate citrate, diphenynhydramine citrate, Chlorpheniramine Maleate, Dextromethorphan Hydrobromide, Oxymetazoline hydrochloride, guaifenesin, ibuprofen, phenylephrine, Acid production control (omeprazole), laxative (loperamide) smoking (nicotine), Ezetimibe, Simvastatin, Eptifibatide, Sitagliptin, Metformin, Losartan Potassium, Hydrochlorothiazide, Finasteride, Enalapril maleate, Hydrochlorothiazide, raltegravir, peginterferon alpha-2b, caspofungin acetate, imipenem and cilastatin sodium, ertapenem sodium, moxifloxacin, posaconazole, Indinavir sulfate, efavirenz, ribavirin USP, peginterferon alfa and ribavirin, rizatriptan benzoate, dorzolamide hydrochloride, Montelukast sodium, infliximab, mometasone furoate monohydrate, desloratadine, etoricoxib, mometasone furoate, golimumab, albuterol sulfate, mometasone furoate/formoterol fumarate, temozolomide, fosaprepitant dimeglumine, Interferon alfa-2b, GARDASIL™, PROQUAD™, MMR II™, VARIVAX™, ROTATEQ™, PNEUMOVAX™, ZOSTAVAX™, alendronate sodium, etonogestrel/ethinyl estradiol, follitropin beta, etonogestrel, desogestrel, Zaleplon, Zolpidem Tartrate, estazolam, flurazepam, temazepam, eszopiclone, zaleplon, zolpidem, Ramelteon, amitriptyline, doxepin, mirtazapine and trazodone, and pharmaceutically active metabolic products and/or metabolic intermediates thereof. In particular embodiments, the pharmaceutical is a sustained release pharmaceutical compound.

Various other compounds are contemplated for use as membrane particles, inclusion in an edible bioreactor disclosed herein, and/or as a product of a bioreaction disclosed herein. For example, antioxidants, hormones and other proteins, enzymes, amino acids, probiotics, etc., may be desirable.

In certain embodiments, hormones are used for hormone replacement and supplementation. Various hormones contemplated for use as a membrane particle include, but are not limited to, adiponectin, aldosterone, androgen, natriuretic peptide, 7-Keto-DHEA, Androstenedione, dehydroepiandrosterone (DHEA), Melatonin, Nor-Androstenedione, pregnenolone, progesterone, 19-Nor-4-Androstenediol, 19-Nor-4-Androstenedione, 19-Nor-5-Androstenediol, 19 Nor-5-Androstenedione, 3-Indolebutyric Acid, 4-Androstenediol, 4-Androstenedione, 6-Furfurylaminopurine, 6-Benzylaminopurine, calcitonin, cortisol, erythropoietin, gonadotropin, human growth hormone (HGH), incretins, leptin, luteinizing hormone, orexin, parathyroid hormone, pregnenolone, progesterone, prolactin, relaxin, renin, testosterone, and vasopressin.

In other embodiments, enzymes and amino acids are contemplated for use as a membrane particle, in an edible bioreactor, and/or as a product of a bioreaction disclosed herein, and include, but are not limited to, alpha galactosidase, amylase, bromelain, cellulase, papain, peptidase, protease, proteolytic enzymes, superoxide dismutase, trypsin, betaine, casein, glutamic acid, L-alanine, L-arginine, L-cysteine, L-glutamine, L-glycine, L-histidine, L-isoleucine, L-leucine, L-lysine, L-methionine, L-ornithine, L-phenylalanine, L-proline, L-taurine, L-threonine, L-tryptophan, L-tyrosine, L-valine, N-acetyl-L-cysteine, protein soluble soy, soy protein isolates, and whey protein isolates.

In certain embodiments, antioxidants contemplated for use as membrane particulates, in an edible bioreactor, and/or as the product of a bioreaction disclosed herein, include, but are not limited to, carotenoids, flavonoids, isoflavones, tocopherol, tocotrienol, lipoic acid, melatonin, superoxide dismutase, coenzyme Q10, alpha lipoic acid, vitamin A, chromium biotin, selenium, and ascorbic acid.

In certain embodiments, carotenoids contemplated for use as membrane particles, in an edible bioreactor, and/or as the product of a bioreaction disclosed herein, include alpha-carotene, beta-carotene, cryptoxanthin, lycopene, lutein, zeaxanthin, apocarotenal astaxanthin, canthaxanthin, lutein/lutein esters, etc.

In some embodiments, flavonoids used as membrane particles, in an edible bioreactor, and/or as the product of a bioreaction disclosed herein, include resveratrol, quercetin, rutin, catechin, proanthocyanins, acai berry extract, raspberry extract, cranberry extract, pomegranate extract, plum extract, cherry extract, rosemary extract, etc.

In some embodiments, isoflavones are used as membrane particles, in an edible bioreactor, and/or as the product of a bioreaction disclosed herein, including, but not limited to, genistein, daidzein, biochanin A, and formononetin.

Further embodiments for particulates in membranes include probiotics to re-establish healthy intestinal bacterial flora. In certain embodiments, probiotics for use in the present disclosure (e.g., as membrane particulates, components in the edible bioreactor such as the active culture, and/or as the product of a bioreaction disclosed herein) include, but are not limited to, Bacillus coagulans GBI-30, 6086, Bifidobacterium animalis subsp. lactis BB-12, Bifidobacterium longum subsp. infantis 35624, Lactobacillus acidophilus NCFM, Lactobacillus paracasei St11 (or NCC2461), Lactobacillus johnsonii (NCC533), Lactobacillus plantarum 299v, Lactobacillus reuteri ATCC 55730 (Lactobacillus reuteri SD2112), Lactobacillus reuteri Protectis (DSM 17938, daughter strain of ATCC 55730), Saccharomyces boulardii, Lactobacillus rhamnosus GR-1 & Lactobacillus reuteri RC-14, Lactobacillus acidophilus NCFM & Bifidobacterium bifidum BB-12, Lactobacillus acidophilus CL1285 & Lactobacillus casei LBC80R, Lactobacillus plantarum HEAL 9 & Lactobacillus paracasei 8700:2, Lactobacillus bulgaricus, Streptococcus thermophiles, and/or Bifidobacterium spp. These probiotics may be distinguishable from bioactive materials, as used herein, including for example certain bacteria cultures referenced herein, as these probiotics generally remain dormant and are not necessarily intended to be raw materials used in a bioreaction. In certain embodiments, probiotics included in the edible bioreactors disclosed herein do not cause, participate in, or contribute to a bioreaction, particularly if the probiotics involvement in the bioreaction renders the product of the bioreaction (e.g., food, beverage, or otherwise) inedible or undesirable.

Plants and plant extracts can provide compositions for dietary supplements, energy products, antioxidants, sleep-aids, weight-loss products, nutraceuticals, oral health compounds, novelty products, etc. Such compositions may be categorized as botanical supplements and botanical extracts. Aqueous or oil based botanical supplements can be combined at low volume with powdered components or be combined into membrane components, edible or potable substances, or into micelles engineered into membranes.

In certain embodiments, botanical extracts and plant-based supplements for use as membrane components include, but are not limited to, Acerola Extracts, Alfalfa, Blue Green algae, Aloe, Amla, Angelica Root, Bacopa monnieri, Mucuna pruriens, Anise Seed, Arnica, Artichoke, Ashwagandha, Astragalus, Ayurvedic Herbs, Barberry, Barley Grass, Barley Sprout Extract, Benzoin, Bilberry, Bioflavonoids, Bitter Melon, Bitter Orange, Black Cohosh, Black Currant, Black Walnut, Bladderwrack, Blue Cohosh, Blueberry, Boswellia, Brahmi, Broccoli, Burdock, Butcher's Broom, Calendula, Capsicum, Cascara Sagrada, Cat's Claw, Catnip herb, Cayenne, Celery Seed, Certified Organic Herbs, Chamomile, Chapparal, Chaste Berry, Chicory Root, Chinese Herbs, Chlorella, Chlorophyll, Citrus Aurantium, Cocoa, Coriander, Corn Silk, Cranberry, Curcuminoids, Damiana, Dandelion, Devilis Claw, Diosgenin, Dong Quai, Echinacea, Elderberry, Elecampane Root, Ephedra, Essential Oils, Eucalyptus, Evening Primrose, Eyebright, Fennel, Fenugreek, Feverfew, Flax Products, Garcinia, Cambogia, Garlic, Gentian, Ginger, Ginkgo, Biloba, Ginseng (American), Ginseng (Panax), Ginseng (Siberian), Goldenseal, Gotu Kola, Grape Seed Extract, Grape Skin Extract, Grapefruit Seed Extract, Green Food Products, Green Lipped Mussel Powder, Green Tea, Griffonia simplicifolia, Guarana, Guggul, Gymnema Sylvestre, Hawthorne, Herbal Extracts, Herbal Teas, Hops, Horehound, Horse Chestnut, Horsetail, Hysop, Ipriflavone, Jojoba Oil, Juniper Berries, Kava Kava, Kelp Extract, Kombucha, Kudzu, Larch, Lavender, Lemon Balm, Licorice Extract, Linden Flowers, Lobelia, Maca, Maitake Mushroom, Marshmallow, Milk Thistle, Molasses, Mushrooms, Neem, Nettle, Noni, Nopal, Oatstraw, Octacosanol, Olive Extract, Orange Peel Extract, Oregano Oil, Oregon Mountain Grape, Organic Sweeteners, Parsley, Passion Flower, Pau d'Arco, Pennyroyal, Peppermint, Pfaffia Paniculata, Pine Bark Extract, Piper longum, Pygeun Africanum, Quercetin, Raspberry Powder, Reishi Mushroom, Resveratrol Extract, Rhubarb Root, Rice Products, Rose Hips, Rosemary Extract, Sage, Sarsaparilla, Saw Palmetto, Schizandra, Seaweed extracts, Senna, Shatvari, Shiitake Mushroom, Silymanrin, Skullcap, Slippery Elm, Soy Isoflavones, Soybean Products, Spirulina, St. John's Wort, Stevia, Summa, Tea Tree Oil, Terminalia arjuna, Tribulus terrestris, Triphala, Turmeric, Uva Ursi, Valerian Extract, Vegetable Extracts, Vitex, Wheat Germ, White Willow Bark, Wild Cherry bark, Wild Yam, Witch Hazel, Wormwood, Yarrow, Yellow Dock, Yerba Santa, Yohimbine, Yucca, 20-ECD 7-9%, Acetyl L-Carnitine HCl 99%, 4-Androstenedione 99%, Adenophora tetraphylla Ext 5:1, Alisma Extract 10:1, Alpha Lipoic Acid 99%, Angelica Root Extract, Arbutin 99%, Artemisia Extract 4:1, Artichoke Extract 5%, Globe Asparagus Extract 4:1, Asparagus Powder, Astragalus Extract 10:1, Astragalus Extract 4:1, Astragalus Extract 5:1, Astragalus Root Extract 0.5%, Astragalus Root Powder, Atractylodes Extract 10:1, Avena sativa Extract 10:1, Avena sativa Extract 4:1, Barbed Skullcap Extract 10:1, Barberry Extract 10%, Bee Pollen Powder, Beta-Sitosterol 35%, Bilberry Extract 10:1, Bitter Melon Extract 8:1, Black Cohosh Extract 2.5%, Black Cohosh Root Powder, Black Pepper Extract 4:1, Black Soy Bean Extract 10:1, Bone Powder, Boswellia Serrata Extract 65%, Broccoli Sprout Extract 10:1, Buchu LeafPowder, Bupleurum (Chai Hu) Extract 5:1, Burdock Root Extract 4:1, Cabbage Extract 4:1, Caffeine (Natural) 86-87%, Caffeine 99%, Calcium Citrate Granular 21%, Calcium-Pyruvate 99%, Carrot Root Extract 4:1, Cassia Nomame Extract 4:1, Catnip Extract 4:1, Cat's Ciaw (Inner Bark), Powder Cauliflower Extract 4:1, Celandine (Greater) Extract 4:1, Celery Seed Extract, Cetyl Myristoleate 11%, Cetyl Myristoleate 20%, Chaenomeles Extract 4:1, Chamomile Flower Extract 10:1, Chamomile Flower Extract 4:1, Chaste Tree Berry Extract 4:1, Chitin Chitosan 80%, Chitosan 90%, Chondroitin Sulfate 90%, Chrysin 99%, Cinnamon Powder, Cistanches Extract 5:1, Citrus Aurantium Extract 6%, Citrus Biotlavonoid Complex 13%, Citrus Peel Extract 5:1, Clove Extract 5:1, Clove Powder, Coca Extract 4:1, Codonopsis pilosula Extract 5:1, Colostrum, Common Peony Extract 8:1, Cordyceps Extract 7%, Cornsilk Extract 4:1, Cornsilk Powder, Corydalis Extract 10:1, Cranberry Extract 4:1, Cranberry Powder, Curcumin Extract 95%, Cuscuta Extract 5:1, Damiana Extract 4:1, Damiana Leaves Powder, Dandelion Powder, Dandelion Root Extract 6:1, Danshen Extract 80%, D-Calcium Pantothenate, Devil's Claw Extract 2.5%, Devil's Claw Extract 4:1, Devil's Claw Root Powder, DHEA 99%, Diosgenin 95%, DL-Phenyl Alanine, DMAE Bitartrate, Dong Quai Extract 10:1, Dong Quai Extract 4:1, Dong Quai Root Powder, D-Ribose, Echinacea angustifolia Extract 4:1, Echinacea Leaf Powder, Echinacea purpurea Extract 10:1, Echinacea purpurea Extract 4%, Echinacea purpurea Extract 4:1, Echinacea purpurea Root Powder, Elder Flower Extract 4:1, Elderberry Extract 20:1, Elderberry Extract 4:1, Epimedium Extract 10%, Epimedium Extract 10:1, Epimedium Extract 4:1, Epimedium Extract 5%, Epimedium Powder, Eucrmmia (Du Zhong) Extract 5:1, Fennel Seed Extract 4:1, Fennel Seed Powder, Fenugreek Extract 4:1, Fenugreek Extract 6:1, Feverfew Extract 5:1, Fisetin, Fish Oil Powder, Forbidden Palace Flower Extract 5:1, Forskolin 8%, Fo-Ti Extract 12:1, Fo-Ti Extract 8:1, Fo-Ti Powder, Gardenia Extract 8:1, Garlic Extract 4:1, Garlic Powder, Gentian Root Extract 6:1, Ginger Extract 4:1, Ginger Root Extract 5%, Ginger Root Powder, Ginkgo biloba Extract 8:1, Ginkgo Extract 24/6%, Ginkgo Extract 24/6%<5, Ginkgo Extract 24/7%, Ginkgo Leaf Extract 4:1, Ginkgo Leaf Powder, Ginseng (Korean) Powder, Ginseng (Panax) Extract 5%, Ginseng (Panax) Extract 8%, Ginseng (Panax) Extract 80%, Glucomannans Konjac Powder, Glucosamine HCl 95%, Granulation Glucosamine HCl 99%, Glucosamine Sulfate Potassium, Glucosamine Sulfate Sodium 95%, Granulation Glucosamine Sulfate Sodium 99%, Goldenrod Extract 4:1, Goldenrod Powder, Goldenseal Root Extract 14%, Goldenseal Root Powder, Gotu Kola Extract 16%, Gotu Kola Extract 4:1, Gotu Kola Extract 8:1, Gotu Kola Powder, Grape Fruit Powder, Grape Seed, Grape Seed Extract 10:1, Grape Seed Extract 20:1, Grape Seed Extract 4:1, Grape Seed Extract 5:1, Grape Seed Extract 95%, Grape Seed Powder, Grape Skin Extract 20:1, Grape Skin Extract 4:1, Grass-Leaved Sweet flag Extract, Green Lip Mussel Extract, Green Tea Extract 30%, Green Tea Extract 4:1, Green Tea Extract 95%, Guarana Seed Extract 10%, Guarana Seed Extract 222%, Guarana Seed Extract 25%, Guggul Extract 10%, Guggul Extract 2.5%, Gugulipid Extract 10%, Gymnerna Sylvestre Extract 25%, Gymnema Sylvestre Powder, Hawthorne Berry Extract 4:1, Hawthorne Berry Powder, Hawthorne Leaf Extract 2%, Herbaceous Peony Extract 5:1, Hesperidin Extract 98%, Honeysuckle Herb Extract 4:1, Hops Flower Extract 4:1, Horehound Extract 10:1, Horehound Extract 4:1, Horehound Herb Powder, Horse Chestnut Extract 20%, Horse Chestnut Extract 4:1, Horse Chestnut Powder, Horsetail Extract 7%, Horsetail Powder, Houttuynia cordata Extract 5:1, Hydrangea Extract 8:1, Hydroxy Apatite, Hyssop Extract 4:1, Indole-3-Carbinol 99%, Isodon Glaucocalyx Extract 10:1, Japanese Knotweed Extract, Jiaogulan Extract 4:1, Jin Qian Cao Extract 4:1, Jing Jie Extract 4:1, Jujube Fruits Extract 4:1, Kava Kava Extract 30%, Kava Kava Powder, Kelp Extract 4:1, Kelp Powder, Kidney Bean Extract 10:1, Kidney Bean Pole 4:1, Kidney Bean Pole 8:1, Kidney Bean Powder, Ko la Nut Extract 10%, Kudzu Extract 4:1, Kudzu Extract 6:1, Lettuce Extract 4:1, L-Glutamine, L-Glycine, Licorice Extract 10%, Licorice Extract 5:1, Licorice Powder, Lotus Leaf Powder, L-Tyrosine, Lycium Fruit Extract 4:1, Lycium Fruit Extract 5:1, Ma Huang Extract 6%, Ma Huang Extract 8%, Maca Extract 0.6%, Maca Root Powder, Magnesium Stearate, Magnolia Bark Powder, Magnolia Officinal Extract 4:1, Maca Extract 4:1, Maitake Mushroom Extract 4:1, Marigold Extract (Lutein 5%), Methoxyisoflavone 99%, Methylsulfonylmethane 99%, Milk Thistle Extract 4:1, Milk Thistle Seed Extract 80% silymarin, Morinda Extract 5:1, Motherwort Extract 4:1, Motherwort Powder, Mucuna pruriens Extract (15% L-Dopa), Muira Puama Extract 12:1, Muira Puama Extract 4:1, Muira Puama Powder, Mushroom Extract 10:1 (reishi), Mustard Seed Extract 8:1, Myrobalan Extract 4:1, Myrrha Gum Extract 2.5%, N-Acetyl-D-Glucosamine, N-Acetyl-L-Cysteine, Nettle Extract 7%, Nettle Leaf Extract 4:1, Nettle Leaf Powder, Noni Powder, Olive Leaf Extract 18%, Olive Powder Orange Peel Extract 4:1, Orange Peel Powder, Oroxylum Indicum Extract 4:1, Oroxylum Indicum Powder, Oyster Meat Powder, Oyster Shell Powder, Papaya Fruit Extract 4:1, Parsley Extract 10:1, Parsley Extract 4:1, Parsley Leaf Extract 4:1, Parsley Powder, Passion Flower Extract 4:1, Passion Flower Powder, Pau D'Arco Powder, Peppermint Extract 4:1, Peppermint Powder, Perilla Seed Extract 4:1, Periwinkle Extract 4:1, Pharbitidis Extract 4:1, Phosphatidyl Serine 20%, Pine Bark Extract 4:1, Plantago asiatica Leaf Extract 5:1, Polygala Tenuifolia Extract 4:1, Polygonum Extract, Polygonum Extract 4:1, Pregnenolone 99%, Propolis Extract 3%, Pseudoginseng Extract, Psyllium extract 4:1, Pumpkin Seed Extract 4:1, Purple Willow Bark Extract 4:1, Purslane Herb Extract 4:1, Pygeum Extract 4:1, Quercetin, Radish Extract 4:1, Radix Isatidis Extract 4:1, Radix Polygoni Extract 4:1, Red Clover Extract 4:1, Red Pepper Extract 4:1, Red Yeast Rice, Red Yeast Rice Extract 10:1, Red Yeast Rice Powder, Rehmannia Root Extract 4:1, Reishi Mushroom Extract 4:1, Rhodiola rosea Extract 4:1, Rhododendron Extract 4:1, Rhododendron Powder, Rhubarb Extract 4:1, Rhubarb Root Powder, Riboflavin (B2), Rice Powder, Rosemary Extract 20%, Rumex Madaid Extract 4:1, Salvia Extract 10:1, Salvia Extract 4:1, SAMe, Saw Palmetto Extract 25%, Saw Palmetto Extract 4:1, Saw Palmetto Extract 45-50%, Saw Palmetto Oil 85-95%, Saw Palmetto Powder, Schizandra Extract 10:1, Schizandra Extract 4:1, Scopolia acutangula Powder, Sea Cucumber Powder, Senna Leaf Powder, Sesame (Black) Seed Powder, Shark Cartilage Powder, Shitake Mushroom Extract, Siberian Ginseng Extract 0.8%, Siberian Ginseng Extract 4:1, Siberian Ginseng Powder, Skullcap Extract 4:1, Skullcap Extract 4:1, Slippery Elm Powder, Sodium-Pyruvate 99%, Songaria cynomorium Extract 4:1, Songaricum Powder, Spirulina Powder, St. John's Wort Extract 0.3%, St. John's Wort Extract 4:1, St. John's Wort Powder, Stanol 50%, Stephania Extract 4:1, Stevia Extract 4:1, Sulfate N+ Suma Root Extract 4:1, Suma Root Powder, Taurine Powder, Thorowax Extract 4:1, Tomato Extract, Tomato Extract (0.2% Lycopene), (trans)-Resveratrol 20-25%, Tribulus Extract 10:1, Tribulus Extract 40%, Tribulus Powder, Triphala Extract 4:1, Turmeric Extract 4:1, Turmeric Root Powder, Uva Ursi Extract 4:1, Uva Ursi Powder, Valerian Root Extract 0.8%, Valerian Root Extract 4:1, Valerian Root Powder, Vinca Major Seed Extract 10:1, White Wax Extract 4:1, White Willow Bark 15% (total salicins), White Willow Bark 20%, White Willow Bark 25%, White Willow Bark Extract 4:1, White Willow Bark Powder, Wild Yam Extract 10:1, Wild Yam Extract 16%, Wild Yarn Extract 4:1, Wild Yam Extract 6%, Wild Yam Powder, Williams Elder Extract 4:1, Wolfberry Fruit Extract 10:1, Wolfiporia Extract 8:1, Yellow Dock Root Extract 4:1, Yerba Mate Extract (2% caffeine), Yerba Mate Extract 4:1, Yohimbe Bark Extract 15:1, Yohimbe Bark Extract 2%, Yohimbe Bark Extract 3%, Yohimbe Bark Powder, and Yucca Extract 4:1.

Nutraceuticals are generally thought of as food or food products that reportedly provide health and medical benefits, including the prevention and treatment of disease, and can be defined as a product isolated or purified from food that is generally sold in medicinal forms not usually associated with food. A nutraceutical may have a physiological benefit or provide protection against chronic disease. Such products may range from isolated nutrients, dietary supplements and specific diets to genetically engineered foods, herbal products, and processed foods such as cereals, soups, and beverages. With recent developments in cellular-level nutraceutical agents, researchers and medical practitioners are developing templates for integrating and assessing information from clinical studies on complementary and alternative therapies into responsible medical practice.

In certain embodiments, nutraceuticals (e.g., particulate nutraceuticals) are used as membrane components, in an edible bioreactor, and/or as the product of a bioreaction disclosed herein, including, but not limited to, 5-Hydroxytryptophan, Acetyl L-Carnitine, Alpha Lipoic Acid, Alpha-Ketoglutarates, Bee Products, Betaine Hydrochloride, Bovine Cartilage, Caffeine, Cetyl Myristoleate, Charcoal, Chitosan, Choline, Chondroitin Sulfate, Coenzyme Q10, Collagen, Colostrum, Creatine, Cyanocobalamin (Vitamin B12), DMAE, Fumaric Acid, Germanium Sesquioxide, Glandular Products, Glucosamine HCL, Glucosamine Sulfate, HMB (Hydroxyl Methyl Butyrate), Immunoglobulin (immune System Support), Lactic Acid, L-Carnitine, Liver Products, Malic Acid, Maltose-anhydrous, Mannose (D-mannose), MSM, Other Carnitine Products, Phytosterols, Picolinic Acid, Pyruvate, Red Yeast Extract, S-adenosylmethionine (SAMe), Selenium Yeast, Shark Cartilage, Theobromine, Vanadyl Sulfate, Velvet Deer Antler, Yeast, ATP, Forskolin, Sterol Esters, Stanol Esters, Probiotics, Lactoferrin, Lutein Esters, Zeaxanthin, Ipriflavone, Isoflavones, Fructo-Oligo-Saccharides, Inulin, Huperzine A, Melatonin, Medicinal Mushrooms, Bile Products, Peptone Products, Glandular Products, Pancreatic Products, Thyroid Products, Ribose, Probiotics, oleo resins, Dill Seed oleoresin, Black Pepper oleoresin, and Capsicum oleoresin.

The various components, including particulates, discussed above may be combined or mixed in or within bioreactors, may be a product of a bioreaction, may complement a product of a bioreaction, may serve as raw materials in a bioreaction, may cause, support, control, or influence a bioreaction, or a combination thereof.

### Edible Bioreactors

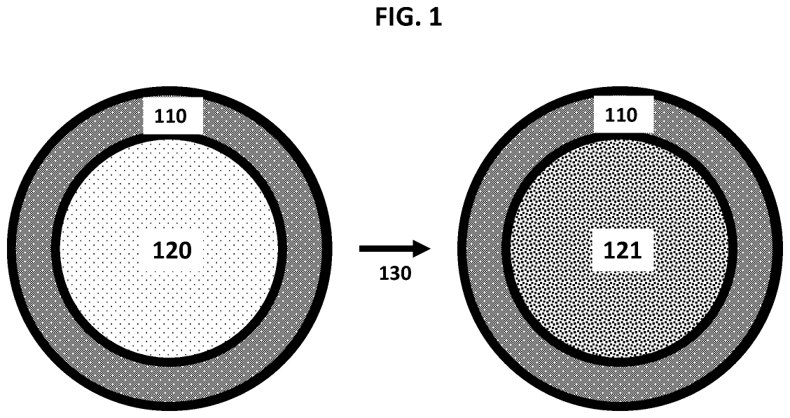

FIG. 1 depicts a diagram of an edible bioreactor. An edible bioreactor comprises an edible membrane 110 wherein the membrane 110 supports a bioreaction 130 or serves as a bioreactor vessel. For example, an edible bioreactor may comprise a core 120 and an edible membrane 110 encapsulating the core 120 wherein a bioreaction 130 occurs in the core 120, 121 while encapsulated in the membrane 110. The core 120 may, for example, comprise a culture and a substrate wherein the culture ferments the substrate to produce edible products in the core 121, including for example, wherein the culture is a yogurt culture and the substrate is milk and the yogurt culture ferments the milk to produce yogurt while encapsulated in the edible membrane. In FIG. 1, the core 120 comprises the substances before or at the initial stage of the bioreaction 130 which subsequently results in a core 121 comprising a product of the bioreaction 130. In this embodiment, the edible membrane 110 does not materially change. In certain embodiments, however, the edible membrane may comprise one or more substances that engage in a process that alters the composition or properties of the edible membrane.

In an exemplary embodiment, a bioreactor comprises an edible membrane and a core wherein the edible membrane encapsulates the core wherein the core has been inoculated before or during encapsulation. A core is inoculated when a culture contacts a substrate wherein the culture and substrate are capable of causing a bioreaction process. In certain embodiments, the edible membrane may encapsulate a core wherein the core is capable of being inoculated. A core is capable of being inoculated if it comprises one or more substances or raw materials wherein at least one of the one or more substances or raw materials is a bioactive substance such as a culture, a substrate that can react with a bioactive substance in a bioreaction, a substance that will experience a bioreaction in response to certain conditions, or a combination thereof. In certain embodiments, a bioreactor comprises a core capable of being inoculated and an edible membrane encapsulating or substantially encapsulating the core before it is inoculated wherein the core is inoculated while encapsulated. A dormant core is an inoculated core where a bioreaction has not started therein.

### Inoculation of Edible Bioreactors

The edible bioreactors disclosed herein may also support one or more upstream processes of a bioreaction, one or more downstream processes of a bioreaction, or a combination thereof. For example, the disclosed edible bioreactor may support inoculation of substances to initiate a bioreaction. In certain embodiments, an edible bioreactor comprises a membrane and a core wherein the core comprises a substrate and the membrane comprises an inoculating substance (e.g., bacteria) wherein the inoculating substance may inoculate the core, an activating substance wherein the activating substance initiate or influence a bioreaction in the core, or a combination thereof.

Inoculation of a core or the initiation of a bioreaction in a dormant core may also occur due to certain environmental conditions (e.g., applying heat, pressure, light, or a combination thereof), physical changes (e.g., squeezing or breaking the bioreactor), adding an inoculating substance to the core, submerging the core into a solution, exposing the core to a gaseous solution, or a combination thereof.

A core may be inoculated while encapsulated or substantially encapsulated in a variety of ways. For example, an edible membrane may encapsulate or substantially encapsulate a core wherein the core comprises a first material. After the core is encapsulated, a second material may be added to the core while it is encapsulated by the edible membrane, for example, through injection, insertion, incision, or a combination thereof, wherein the second material inoculates the core. An edible bioreactor may comprise a core and an edible membrane encapsulating the core wherein the edible membrane comprises a material that inoculates the core. Similarly, an edible bioreactor may comprise a core and an edible membrane encapsulating the core wherein the core comprises a material that inoculates the edible membrane.

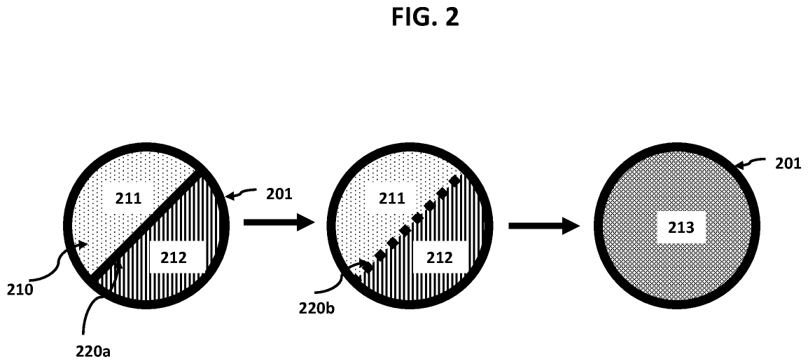

A core may comprise a culture and a substrate wherein the culture has not inoculated the substrate at the start of a time period and wherein the culture inoculates the substrate at the end of the time period. For example, as illustrated in FIG. 2, a core 210 may comprise a culture 211 and a substrate 212 that wherein the culture and the substrate do not interact or have minimal interaction due to, for example, a barrier 220a wherein the permeability of the barrier 220a changes over time due to evaporation, sublimation, degradation, mixing, dissolution, reacting, melting, freezing, condensation, deposition, or a combination thereof. The permeability of the barrier 220a may also depend on the physical characteristics of the barrier 220a, including for example its thickness, density, physical arrangement, or a combination thereof. For example, the thickness of a semi-permeable barrier will affect the time required for a substance to permeate the barrier so the barrier may be designed to be thicker to increase the time before a culture and a substrate interact.

In an exemplary embodiment, the barrier 220a may react with the core 210, including for example, disintegrating, combining, absorbing, or dissolving entirely into the core 210. For example, at the start of a time period, the barrier 220a comprises an impermeable solid such as ice or other frozen liquid, an oil, or a wax. As the barrier 220b changes into a liquid state, for example as ice melts into liquid water, the barrier 220b becomes more permeable, allowing the culture 211 and the substrate 212 to interact in a bioreaction. The core 210 absorbs the barrier 220b as the barrier 220a changes into its liquid state. In preferred embodiments, a barrier 220a may comprise of an edible substance that has a melting point near or around room temperature, including for example, coconut oil, palm oil, and certain waxes. In some embodiments, the core 211, 212 and the barrier 220a react, combine, or mix completely so that the core 213 no longer comprises a barrier.

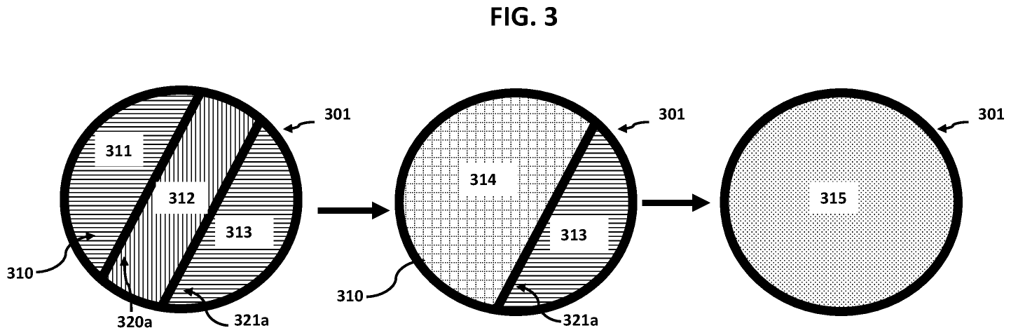

FIG. 3 depicts an embodiment where an edible bioreactor comprises a plurality of barriers to initiate two reactions at different times wherein one of the reactions is preferably a bioreaction. Specifically, an edible bioreactor comprising a core 310 and an edible membrane 301 encapsulating the core 310 wherein the core 310 comprises a first substance 311, a second substance 312, a third substance 313, a first barrier 320a and a second barrier 321a. In this example, the first barrier 320a prevents the first substance 311 from contacting the second substance 312 or the third substance 313 and the second barrier 321a prevents the third substance 313 from contacting the first substance 311 or the second substance 312. The first barrier 320a and the second barrier 321a comprise different substances, substances with the same composition at different ratios, the same substance with different physical properties, including for example, density, weight, size, shape, or a combination thereof.

Preferably, the first barrier 320a and second barrier 321a become permeable in response to different conditions, at different times, or a combination thereof. For example, the first barrier 320a becomes permeable at the end of a first time period. As a result, the first substance 311 interacts with the second substance 312, including for example, wherein the first substance 311 and second substance 312 interact in a bioreaction that results in a first product 314. The second barrier 321a becomes permeable at the end of a second time period wherein the second time period is longer than the first time period. Between the end of the first time period and end of the second time period, the second barrier 321a prevents the third substance 313 from contacting the first product 314. At the end of the second time period, the second barrier 321a becomes more permeable so that the third substance 313 and the first byproduct 314 interact, including for example, in a bioreaction. The result of this interaction is second product 315.

The first barrier 320a and the second barrier 321a may comprise of the same material, for example, ice or other frozen liquid, coconut oil, palm oil, or any edible substance that is impermeable as a solid. The first barrier 320a, however, may be more permeable than the second barrier 321a because the first barrier 320a has less volume or thickness than the second barrier 321a, the first barrier 320a may further comprise a solvent that accelerates melting, or a combination thereof. Similarly, the first barrier 320a and the second barrier 321a may each comprise different materials or different compositions of the same or similar materials so that the first barrier 320a becomes permeable before the second barrier 321a. The differences between the first barrier 320a and the second barrier 321a, including for example differences in materials, composition, size, properties, or combination thereof, may cause the first barrier 320a to melt before the second barrier 321a.

For example, a first barrier 320a comprising ice and a second barrier 321a comprising palm oil will change into a liquid state to become permeable in different conditions, including for example, temperature. The first barrier 320a and the second barrier 321a may each comprise of the same material, including for example ice, wherein the first barrier 320a is twice the thickness of the second barrier 321a. Although each barrier 320a, 321a comprises ice, the first barrier 320a will becoming permeable more quickly than the second barrier 321a because it will take longer for the ice in the second barrier 321a to melt so that it is permeable.

In other embodiments, a barrier becomes permeable in response to a biochemical condition. For example, a barrier may comprise a substance that becomes permeable upon exposure to an appropriate enzyme, which may be present due to an earlier bioreaction or by direct introduction into the core. In this example, a barrier may comprise an enzyme-degradable polymer, including for example, a starch or cellulose.

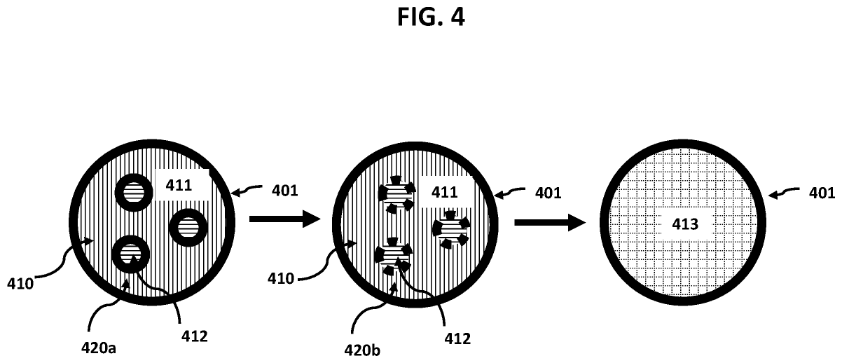

FIG. 4 is a diagram of an edible bioreactor comprising a core 411 and an edible membrane 401 encapsulating the core 411 wherein the core 411 comprises a plurality of core units 412. Each core unit 412 in the plurality of core units 412 may be encapsulated by one or more edible membrane units 420a. In certain embodiments, the one or more membrane units 420a are barriers that prevent the encapsulated core unit 412 from contacting one or more substances of the core 411. The barrier membrane units 420a become permeable in response to certain conditions, a duration of time, or a combination thereof. When the barrier membrane unit 420a becomes permeable, the encapsulated core unit 412 contacts the one or more substances in the core 411. In certain embodiments, the plurality of core units 412 may contain a first core unit and a second core unit wherein the first core unit and the second core unit comprise different materials or the materials with different compositions, different weights, different sizes, or a combination thereof.

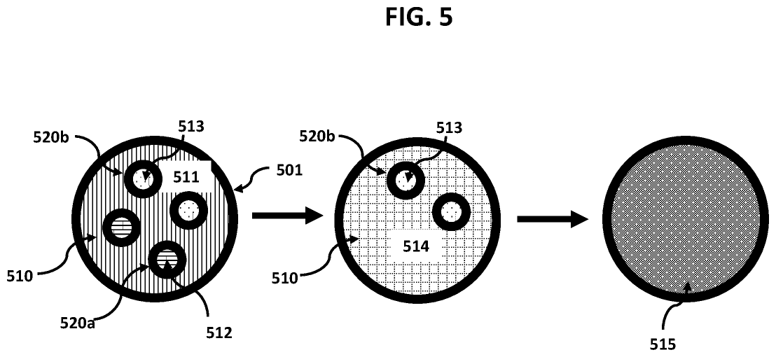

FIG. 5 is a diagram of an exemplary embodiment of an edible bioreactor comprising a plurality of barrier membrane units 520 wherein the plurality of barrier member units 520 comprises a first barrier membrane unit 520a encapsulating a core unit 512 and a second barrier membrane unit 520b encapsulating a second core unit 513 wherein the first barrier membrane unit 520a and the second barrier membrane unit 520b become permeable at different times due to different materials, compositions, physical properties such as volume, density, and mass, chemical properties, or a combination thereof. Likewise, the first barrier membrane unit 520a and second barrier membrane unit 520b may become permeable in response to different conditions, including physical conditions like temperature or chemical conditions such a pH.

Referring to FIG. 5, the core 511 may comprise a substrate and a plurality of core units 512, 513 wherein a bioreaction occurs when one or more of the plurality of core units 512, 513 contacts the substrate 511. The core 511 may further comprise a plurality of barrier membrane units 520 comprising a first barrier membrane unit 520a and a second barrier membrane unit 520b wherein the first membrane unit encapsulates a first core unit 512 in the plurality of core units and the second barrier membrane 520b encapsulates a second core unit 513 in the plurality of core units wherein the first barrier membrane unit 520a becomes impermeable at a different time or in response to different conditions than the second barrier membrane unit 520b. In some embodiments, the second barrier membrane unit 520b becomes impermeable in response to a product created or produced, or conditions caused by, the first core unit 520a interacting with a substance in the core 511.

In certain embodiments, an edible bioreactor comprises a core and an edible membrane encapsulating the core wherein the core may comprise a culture and a substrate wherein the culture has not contacted the substrate. In these embodiments, the culture contacts the substrate in response to a change in the environmental conditions, including for example, temperature, pressure, light, pH, or a combination thereof. The core inoculates the substance upon contact. The inoculation may also result from a chemical reaction by introducing a substance into the core, submerging the core into a solution, exposing the core to a gaseous composition, or a combination thereof. In some instances, an inoculated core may remain dormant until activated in response to environmental conditions, chemical reactions, or a combination thereof.

It is understood that endless differences between or among two or more barriers exist due to myriad materials, compositions, properties, conditions, or combination thereof that may be selected for each barrier and the examples herein do not limit the scope of the present disclosure.

### Exemplary Products Using Edible Bioreactors

An edible bioreactor may produce a variety of bioreaction products (or alternatively “payloads”) that result from one or more bioreactions in an edible bioreactor. For example, the bioreaction products may be fermented foods or beverages such as alcohol (e.g., beer or wine), yogurt, kefir, cheese, sauerkraut, etc. Tissue may also be derived from culturing plant or animal cells in an edible bioreactor. For example, an edible bioreactor may be used to grow plant or animal cells to create and grow edible tissues, including for example, culturing bovine, chicken, or other animal cells to make cell-based meat. In sum, an edible bioreactor may be used to grow a broad range of cells, including bacteria, fungi (e.g., yeast), plant cells, animal cells, or a combination thereof.

An edible bioreactor may also serve as a vessel for acellular bioreactions, i.e., encapsulating biochemically-active materials that are not cells, including for example, an enzyme and a substrate that reacts with the enzyme (e.g., cellulose+beta-glucosidase→glucose). Although making supplements and other ingestible products containing enzymes or bacteria (e.g., probiotics) is well known in the art, these enzymes and bacteria are typically intended to remain dormant through the entire duration production and ingestion processes. In the present disclosure, however, the enzymes and bacteria can be raw materials and/or engage in an active bioreaction process when they are in, within, or part of the edible bioreactor or a composition therein.

The product may take multiple forms and have a variety of physical properties. For example, the viscosity of a core comprising a bioreaction product may range from liquid beverages (kombucha, kefir, beer, etc.) to solids (fermented vegetables, cheese, hydrogel scaffolds for plant or mammalian cell growth, etc.), or be somewhere in between (yogurt, condiments, etc.). The products may also comprise one or more gases.

A bioreactor may also produce bioreaction byproducts which generally means any secondary or undesired materials produced in addition to the desired bioreaction products. As used herein, depending on context, bioreaction products may include byproducts or may be mutually exclusive of byproducts.

As discussed below, in certain embodiments, an edible membrane preferably has properties that account for or are adapted to discard, release, separate, eliminate, or process one or more bioreaction byproducts, including for example, where the one or more bioreaction byproducts comprises a gaseous byproduct (e.g., carbon dioxide). In certain embodiments, an edible bioreactor may support one or more downstream processes after a bioreaction. For example, a bioreactor for yogurt may comprise an edible membrane that enables straining of the bioreaction product, the coagulated proteins, to remove excess liquid, a bioreaction byproduct, to increase the viscosity of the final product.

### Edible Bioreactors for Cores Releasing Gaseous Byproducts

A bioreaction may produce a product and one or more byproducts wherein the one or more byproducts may be a solid, liquid, semi-solid, gas, or combination thereof. For edible bioreactors that contain a core that produces a gas byproduct (including for example, edible bioreactors that contain and/or support a bioreaction to produce kombucha, kefir, beer, fermented vegetables, etc.), the membrane of the bioreactor is preferably gas permeable, of sufficient strength to withstand the pressure caused by the gaseous byproduct (e.g., carbon dioxide), or semi-permeable. In certain embodiments, the membrane of the bioreactor is selectively permeable, e.g., substantially permeable to gas, and substantially impermeable to liquid. The permeability of the membrane and its strength to withstand pressure caused by a gaseous byproduct may depend on multiple factors, including for example, the composition of the membrane, interactions between the membrane and substances or materials contacting the membrane (e.g., the membrane may comprise of polymers that crosslink with polymers in a core that the membrane encapsulates or a second membrane may comprise of polymers that crosslink with polymers in the first membrane).

Similarly, an edible bioreactor that can release gaseous byproducts may be placed in an environment where the conditions are selected to minimize or control the gaseous byproduct to ensure, for example, that the membrane stays intact. For example, a pressure-controlled environment, chamber, or outer shell can be utilized to maintain sufficient pressure on the surface of the membrane of the edible bioreactor so that it stays intact during a bioreaction that releases a gaseous byproduct.

The edible core can be formed in different ways, preferably in a manner that depends on composition of the core, including characteristics thereof. For example, because many microbes can survive being frozen, an edible core comprising bacteria wherein the bacteria will cause or be part of a bioreaction, the core can be frozen to permit encapsulating the core with techniques known to coat a solid. Alternatively, for cores that cannot be solidified, the core may be encapsulated at room temperature, for example, if the core has sufficient fibers to enable gelling in a mold. For edible bioreactors that support bioreactions involving plant or animal cells, the bioreactors are preferably either made and inoculated at room temperature within a gel or a substance that is semi-solid or frozen following the inclusion of appropriate cryoprotectants such as glycerol or sugars to prevent ice crystal formation because such cells may otherwise burst when frozen.

The exemplary examples that follow demonstrate certain embodiments of the disclosure.

### Preparation of Fermented Milk Products

In one embodiment of edible bioreactors, an edible bioreactor may support the fermentation of milk to yield edible products (e.g., foods) such as yogurt, kefir, and cheese (e.g. soft cheese) with an edible membrane that encapsulates a core comprising milk and the appropriate microbial cultures.

### Example 1—Preparation of Yogurt Edible Bioreactors

In this example, milk was inoculated with mesophilic lactic acid bacteria, encapsulated into an alginate membrane to form an edible bioreactor, and then fermented to produce yogurt using these formulations.

Plain Yogurt Inner Liquid

| Ingredient | Mass per 100 g |
|---|---|
| Pasteurized/homogenized whole cow milk | 94.3 g |
| Yogurt (containing live and active cultures) | 5.7 g |

2% Alginate Membrane

| Ingredient | Mass per 100 g |
|---|---|
| Water | 98.0 g |
| Sodium alginate | 2.0 g |

Chitosan Crosslinking Bath

| Ingredient | Mass per 100 g |
|---|---|
| Water | 86.8 g |
| Chitosan | 4.7 g |
| Lactic Acid | 5.5 g |
| Calcium Lactate | 3.0 g |

The yogurt edible bioreactor was prepared by the following process.

1) Milk was heated to 85° C. and maintained at this temperature for 30 min. This step functions dually to sterilize the milk and denature the proteins, increasing viscosity and improving texture.

2) Upon cooling down to room temperature, milk was inoculated with a small quantity of yogurt containing active lactic acid bacterial cultures by mixing.

3) The inoculated liquid was frozen into the desired shape using an appropriate mold, to form a frozen core. In this case, liquid was frozen in 8.5 mL spherical silicone molds.

4) The frozen core was submerged in liquid nitrogen to obtain a super-frozen material.

5) The core was next submerged in an alginate bath, creating a coating of frozen alginate on the surface.

6) The coated core was then submerged in a room temperature chitosan bath until the frozen core had completely thawed to allow sufficient time for crosslinking of the alginate coating. At this point, the sphere was dried by gently rolling around on a paper towel.

7) Finally, the edible bioreactors were incubated at the appropriate temperature (in this example, room temperature for the mesophilic bacterial cultures) until the inner liquid had been successfully fermented into yogurt.

8) When fermentation was complete, the edible bioreactors were stored in a refrigerator in sealed containers to prevent further fermentation.

The resulting pH of the inner liquid of the edible bioreactors showed that a bioreaction occurred. Lactic acid bacteria growth is associated with the conversion of lactose to lactic acid which concomitantly reduces pH. Viscosity also increases as the milk proteins coagulate. Successful fermentation within the edible bioreactors is thus demonstrated by a decrease in pH of inner liquid, accompanied by a substantial increase in viscosity.

Inner Liquid pH

| Timepoint | pH (w/o cultures) | pH (with cultures) |
|---|---|---|
| 1 h | 6.52 ± 0.04 | 6.37 ± 0.05 |
| 24 h | 6.33 ± 0.04 | 4.59 ± 0.09 |
| 48 h | 5.99 ± 0.00 | 4.38 ± 0.08 |

### Example 2—Whey Byproduct Removal from Yogurt Edible Bioreactors

As an alternative to adding ingredients like tapioca starch, yogurt can be thickened by removing whey. Traditionally, this is accomplished by straining the yogurt product through fabric, such as a cheesecloth. In this example, yogurt edible bioreactors are designed to feature membranes with increased water permeability, such that excess liquid can readily be filtered from the yogurt contained within the bioreactor. This is a significant advantage over traditional methods.

The water permeability rate of the edible bioreactor membranes can be tuned, such as by adjusting the composition of either the alginate bath or the crosslinking bath. In this example, moisture loss data is presented for plain yogurt cores encapsulated by one of two different alginates.

A peanut butter alginate was formulated as follows.

Peanut Butter Alginate Recipe

| Ingredient | Amount |
|---|---|
| Water | 197.1 g |
| Unsalted Peanut Butter (Teddie) | 60.0 g |
| Cane Sugar | 15.0 g |
| Sodium Alginate | 3.0 g |
| Pectin | 3.0 g |
| Inulin | 15.0 g |
| Peanut Butter Flavor UA3735 | 0.9 g |
| 2x White Chocolate Flavor UB5948 | 4.0 g |
| GNT EXBERRY Rustic Brown Color #931804 | 3.0 g |

These bioreactors were crosslinked in one of two different crosslinking baths: the chitosan crosslinking bath described previously and the calcium crosslinking bath outlined below. These yogurt edible bioreactors were prepared using methods described herein. Following the completion of yogurt fermentation, the bioreactors were stored in a refrigerator and weighed each day to determine the moisture loss.

Moisture loss in this example is reported below as the percentage of mass remaining at each timepoint as compared to when the bioreactors were first placed in the fridge.

Plain Yogurt Edible Bioreactor Moisture Loss

| Timepoint | Residual Mass (chitosan) | Residual Mass (calcium) |
|---|---|---|
| 0 d | 100.0% | 100.0% |
| 1 d | 98.2% | 86.3% |
| 2 d | 97.4% | 81.9% |
| 4 d | 97.2% | 77.9% |

Peanut Butter Yogurt Edible Bioreactor Moisture Loss

| Timepoint | Residual Mass (chitosan) | Residual Mass (calcium) |
|---|---|---|
| 0 d | 100.0% | 100.0% |
| 1 d | 97.4% | 90.5% |
| 2 d | 95.7% | 89.9% |
| 4 d | 95.6% | 86.1% |

Yogurt edible bioreactors crosslinked without the use of chitosan readily expelled visible quantities of liquid. After four days, the yogurt in the cores of these materials were substantially more viscous than their chitosan-coated counterparts and exhibited the texture of Greek-style yogurt. When the desired core consistency was achieved, moisture loss from the calcium-crosslinked yogurt edible bioreactors could be abruptly halted at any timepoint by simply enrobing the spheres in a second layer of alginate followed by crosslinking for 2 minutes in the chitosan crosslinking bath.

The particulates used for the edible bioreactor can advantageously affect the membrane strength, diffusion, permeability, and stability. For example, a core fermented in a bioreactor comprising a membrane that was crosslinked using chitosan resulted in a bioreaction byproduct that had a thinner consistency compared to the cores cultured with membranes crosslinked with only calcium. If the desired byproduct is a thicker yogurt (e.g., Greek-style yogurt), it is preferable to use membranes comprising only calcium.

The environmental conditions (temperature, humidity, pressure, etc.) in which the bioreactor is placed while bioreactions occur can also affect the reaction and the resulting bioreaction product. Certain data collected in this example indicate that samples cultured in an open atmosphere decrease in mass at different rates than samples cultured in controlled atmospheric conditions. The bioreaction can also be affected by a combination of the membrane composition and environmental conditions. For example, samples crosslinked with chitosan retain more moisture than their calcium-crosslinked counterparts.

### Preparation of Water Kefir, Teas, and Alcoholic Beverages

In these examples, exemplary embodiments of edible bioreactors to produce liquid beverages using fermentation drinks such as water kefir, kombucha, and alcoholic beverages are described.

### Example 3—Preparation of Kombucha Edible Bioreactors

In this example, black tea which had been fermented once was encapsulated in an alginate membrane along with sugar and flavors to undergo a second fermentation to produce a flavored kombucha beverage.

Below are the formulations used in this example.

Raspberry Lime Kombucha Inner Liquid

| Ingredient | Mass per 100 g |
|---|---|
| Kombucha (containing live and active cultures) | 90.1 g |
| Cane sugar | 3.2 g |
| Raspberry puree | 3.2 g |
| Calcium lactate | 2.0 g |
| Lime juice | 1.5 g |

2.25% Alginate Membrane

| Ingredient | Mass per 100 g |
|---|---|
| Water | 97.75 g |
| Sodium alginate | 2.25 g |

A kombucha edible bioreactor was prepared by the following process.

1) The liquid ingredients were mixed together and the sugar and calcium lactate were incorporated by blending.

2) The inoculated liquid was frozen into the desired shape using an appropriate mold. In this case, liquid was frozen in 8.5 mL spherical silicone molds.

3) A frozen core was submerged in liquid nitrogen to obtain a super-frozen material.

4) The super-frozen core was next submerged in an alginate bath, creating a coating of frozen alginate on the surface.

5) The coated core was then submerged in a room temperature chitosan bath until the frozen core had completely thawed to allow sufficient time for crosslinking of the alginate coating. At this point, the sphere was dried by gently rolling around on a paper towel.

6) A second coating was applied by submerging the sphere in the alginate bath a second time, followed by crosslinking in a chitosan bath for another 2 minutes and subsequent drying on a paper towel.

7) Finally, the edible bioreactor was incubated at room temperature until the inner liquid had been successfully fermented.

8) When fermentation was complete, the edible bioreactors were stored in a refrigerator in sealed containers to prevent further fermentation.

Successful secondary kombucha fermentation was accompanied by several observable changes, including a modest reduction in pH, substantial carbon dioxide (CO₂) production, and rapid proliferation of yeast cells. Given the gas permeability of these alginate membranes, CO₂ production could be measured by collecting the gas released over a solution of HCl with a pH of ˜4.0 in an inverted graduated cylinder.

An acidic solution was utilized to minimize the solubility of CO₂ in water, which would otherwise result in an underreporting of the quantity of CO₂ produced. The observed volume of CO₂ released was converted to mass assuming a density of 1.98 mg/mL at standard temperature and pressure.

The cumulative production of CO₂ over a period of three weeks is tabulated below for the fermentation of raspberry lime kombucha in edible bioreactors.

CO₂ Production

| Timepoint | Cumulative CO₂ Produced |
|---|---|
| 0 d | 0.00 mg/g kombucha |
| 1 d | 0.02 mg/g kombucha |
| 4 d | 0.02 mg/g kombucha |
| 5 d | 0.08 mg/g kombucha |
| 6 d | 0.12 mg/g kombucha |
| 7 d | 0.12 mg/g kombucha |
| 8 d | 0.12 mg/g kombucha |
| 11 d | 0.40 mg/g kombucha |
| 12 d | 0.71 mg/g kombucha |
| 13 d | 1.11 mg/g kombucha |
| 14 d | 1.44 mg/g kombucha |
| 15 d | 1.86 mg/g kombucha |
| 19 d | 2.97 mg/g kombucha |
| 20 d | 3.05 mg/g kombucha |
| 22 d | 3.47 mg/g kombucha |

This example demonstrates edible bioreactors may extend to other bioreactions beyond those facilitated by bacteria alone, e.g., using a combination of bacteria and fungi (e.g., yeast). In addition, it illustrates the utility in formulating a membrane of the edible bioreactor so that the membrane has a permeability conducive to the given bioreaction.

### Example 4—Preparation of Kombucha Edible Bioreactors with Gelled Cores

In addition to the liquid-core kombucha edible bioreactors described above, kombucha edible bioreactors with weakly gelled cores were prepared by the exclusion of calcium lactate, demonstrating an alternate texture. In this example, the alginate coating is only crosslinked from the outside-in (direct spherification), rather than from both the outside-in and the inside-out (direct spherification and reverse spherification). This resulted in the diffusion of some alginate material into the liquid core and subsequent gelation with the citric acid present in both the raspberry puree and lime juice. Thus, the core consistency of edible bioreactor cores can be controlled by the presence or absence of calcium lactate and citric acid. The inner liquid formulation for this example is presented below.

Raspberry Lime Kombucha Inner Liquid (gelled cores)

| Ingredient | Mass per 100 g |
|---|---|
| Kombucha (containing live and active cultures) | 92.1 g |
| Cane sugar | 3.2 g |
| Raspberry puree | 3.2 g |
| Lime juice | 1.5 g |

Regardless of the inner liquid consistency, fermentation appeared unaltered. For this example, the bioreaction was monitored by enumeration of yeast cells under a light microscope using a Petroff-Hausser counting chamber for a duration of one week.

Yeast Proliferation

| Timepoint | Cellular Density |
|---|---|
| 0 days | 1.95 × 10⁶ cells/mL kombucha |
| 3 days | 9.94 × 10⁶ cells/mL kombucha |
| 4 days | 14.3 × 10⁶ cells/mL kombucha |
| 5 days | 16.1 × 10⁶ cells/mL kombucha |
| 6 days | 17.0 × 10⁶ cells/mL kombucha |
| 7 days | 21.6 × 10⁶ cells/mL kombucha |

### Fermented Fruits, Vegetables, and Condiments

In another embodiment of edible bioreactors, fruits and vegetables can be preserved or transformed into condiments via lacto-fermentation through the inclusion of appropriate microbial cultures.

### Example 5—Preparation of Sauerkraut Edible Bioreactors

In one example, chopped cabbage was encapsulated in an alginate membrane along with salt and bacterial cultures to produce sauerkraut.

Below are the formulations used in this example.

Sauerkraut Inner Liquid

| Ingredient | Mass per 100 g |
|---|---|
| Water | 56.5 g |
| Chopped cabbage | 41.5 g |
| Salt | 2.0 g |
| Lyophilized lactic acid bacterial cultures | 25 mg |

2% Alginate Membrane

| Ingredient | Mass per 100 g |
|---|---|
| Water | 84.0 g |
| Inulin | 12.0 g |
| Sodium alginate | 2.0 g |
| Pectin | 2.0 g |

A sauerkraut edible bioreactor was prepared by the following process.

1) Chopped cabbage, water, and salt were mixed. Lyophilized lactic acid bacterial cultures were added to inoculate the mixture.

2) The inoculated liquid was frozen into the desired shape using an appropriate mold. In this case, liquid was frozen in 8.5 mL spherical silicone molds.

3) A frozen core was submerged in liquid nitrogen to obtain a super-frozen material.

4) The core was next submerged in an alginate bath, creating a coating of frozen alginate on the surface.

5) The coated core was then submerged in a room temperature chitosan bath until the frozen core had completely thawed to allow sufficient time for crosslinking of the alginate coating. At that point, the sphere was dried by gently rolling around on a paper towel.

6) The edible bioreactors were incubated at room temperature until the inner liquid had been successfully fermented.

7) When fermentation was complete, the edible bioreactors were stored in a refrigerator to prevent further fermentation.

Vegetable lacto-fermentation is associated with the production of lactic acid and CO₂. As a result of the high gas-permeability of these alginate membranes, the CO₂ produced by the lactic acid bacteria was prevented from building up inside the bioreactors, which would otherwise lead to increased pressure and potential membrane rupture.

Successful fermentation was evidenced by a rapid reduction in pH over the course of three days, and is presented below.

Inner Liquid pH

| Timepoint | pH |
|---|---|
| 0 days | 5.65 ± 0.16 |
| 1 day | 4.83 ± 0.03 |
| 2 days | 4.66 ± 0.05 |
| 3 days | 3.90 ± 0.01 |

### Embodiments

A number of embodiments have been described. Nevertheless, it will be understood that various modifications may be made without departing from the spirit and scope of the present disclosure.

For example, the edible bioreactors can include ingestible substances contained in a soft membrane; ingestible substances contained in a soft membrane inside a hard edible shell; multiple membrane-enclosed servings dispersed in a hard edible shell; and multiple membrane-enclosed servings dispersed in a hard biodegradable shell. The exemplary edible bioreactors discussed above are generally 5-6 cm in diameter, but edible bioreactors having other diameters are also contemplated, for example, edible bioreactors with a diameter of 7-8 cm, or smaller edible bioreactors with “grape” membranes having diameters of 1-3 cm.

In some embodiments, edible bioreactors include a poly(lactic acid) (PLA) outer shell and use inner membranes ranging from the sodium alginate membranes to edible waxes of the kinds used on fine chocolates occasionally. The latter have a distinct advantage of repelling water. Some embodiments may contain one or more combinations of such materials as “shells” or “membranes”, for example, a sodium alginate membrane, hardened/cured with calcium, may be covered with an edible wax and then placed within a PLA shell.

In some embodiments, multiple inner containers can be protected by a single outer shell. For example, in some embodiments, a shell of PLA is filled with ‘grapes’ of liquid that are bioreactive and closed up like a bottle. The outer shell can be opened and the ‘grapes’ consumed with the liquid they contain including, e.g., the product of the bioreaction. The outer shell is biodegradable and the advantage of the inner membranes is to reduce direct contact of the bottle and the bioreactive core and therefore avoid degradation of the bottle itself.

Selected illustrative embodiments of the machines and compositions are described above in some detail. It should be understood that only the essential machine components, ingredients and/or formulations which are considered necessary for clarifying the exemplified embodiments have been described herein. Other machine components, ingredients, and/or formulations equivalents are assumed to be known and understood by those skilled in the art. Moreover, while working examples of machine components, ingredients, and/or formulations have been described, the present disclosure is not limited to the working examples described above, but various design alterations may be carried out without departing from the machine components, ingredients, and/or formulations as set forth in the claims.

1. An edible bioreactor, comprising (e.g., consisting of) an edible core and an edible membrane encapsulating the core, wherein the edible membrane supports a bioreaction in the edible core.

2. An edible bioreactor, comprising (e.g., consisting of) an edible core, a first edible membrane encapsulating the edible core, and a second edible membrane encapsulating the first edible membrane, wherein:
(i) the first edible membrane inoculates the edible core (or a component within the edible core, such as an active culture) and the second edible membrane supports a bioreaction; or
(ii) the edible core inoculates the first edible membrane (or a component within the first edible membrane, such as an active culture) and the second edible membrane supports a bioreaction.

3. The edible bioreactor of claim 1, wherein the edible membrane,
first edible membrane, and/or second edible membrane comprises an edible polymer.

4. The edible bioreactor of claim 1, wherein the edible membrane,
first edible membrane, and/or second edible membrane comprises edible particles.

5. The edible bioreactor of claim 1, wherein the edible membrane, first edible membrane, and/or second edible membrane comprises a plurality of edible polymers.

6. The edible bioreactor of claim 1, wherein the edible membrane, first edible membrane, and/or second edible membrane forms a bioreactor vessel.

7. The edible bioreactor of any one of the preceding claims, wherein the edible membrane, first edible membrane, and/or second edible membrane supports an upstream process, a downstream process, or both an upstream process and a downstream process of a bioreaction in the edible core.

8-9. (canceled)

10. The edible bioreactor of claim 1, wherein the edible membrane inoculates the edible core, or a component within the edible core (e.g., an active culture).

11. The edible bioreactor of claim 1, wherein the edible membrane, first edible membrane, and/or second edible membrane is substantially impermeable or selectively permeable, e.g., permeable to gas but impermeable to liquid and/or solid to liquid.

12. (canceled)

13. The edible bioreactor of claim 1, wherein the bioreaction occurs at a temperature between about 15° C. and about 50° C., e.g., about 20° C., about 25° C., about 30° C., about 35° C., about 40° C., or about 45° C.

14. (canceled)

15. The edible bioreactor of claim 1, wherein a component (e.g., a liquid) within the edible core has a pH less than 7.0 (e.g., between about 6.0 and 6.9, e.g., about 6.5, 6.4, 6.3, 6.2, or 6.1), and optionally wherein the pH is reduced during the bioreaction, e.g., to less than 6.0, e.g., between about 3.0 and 6.0, between about 3.0 and 4.0, between about 4.0 and about 5.0, between about 5.0 and about 6.0, about 5.5, about 5.0, about 4.5, about 4.0, about 3.5, or about 3.0.

16. The edible bioreactor of claim 1, wherein the bioreaction comprises a fermentation, e.g., a bacterial fermentation.

17. (canceled)

18. The edible bioreactor of claim 1, wherein the edible core comprises an active culture.

19. The edible bioreactor of claim 1, wherein the edible membrane, first edible membrane, and/or second edible membrane comprises an active culture.

20-21. (canceled)

22. The edible bioreactor of claim 1, wherein the edible core and/or the edible membrane comprises a substrate that can be fermented by an active culture.

23-25. (canceled)

26. The edible bioreactor of claim 1, wherein the product of the bioreaction is an edible product (e.g., a food, beverage, supplement, or pharmaceutical).

27. (canceled)

28. The edible bioreactor of claim 1, wherein the edible membrane, the first edible membrane, and/or the second edible membrane comprises an alginate (e.g., sodium alginate).

29. (canceled)

30. The edible bioreactor of any one of the preceding claims, wherein the edible core comprises a liquid, solid, semi-solid, foam, or a combination thereof, and is optionally frozen.

31. (canceled)

32. The edible bioreactor of claim 1, wherein the edible core comprises an edible barrier.

33. (canceled)

34. A process for making an edible bioreactor of any one of the preceding claims, comprising the steps of:
(i) combining a substrate and an active culture to make an edible core component;
(ii) freezing the edible core component to provide a frozen edible core component;
(iii) optionally, shaping the frozen edible core component, e.g., into a sphere;
(iv) submerging the frozen edible core component in liquid nitrogen to obtain a super-frozen edible core component;
(v) submerging the super-frozen edible core component in an edible membrane precursor (e.g., an alginate bath), thereby encapsulating the super-frozen edible core component with the edible membrane, to provide an encapsulated edible core;
(vi) optionally, submerging the encapsulated edible core in a second edible membrane precursor, to provide an encapsulated edible core comprising multiple edible membranes;
(vii) submerging the encapsulated edible core of step (v) or (vi) in a crosslinking bath (e.g., a chitosan crosslinking bath, or a calcium crosslinking bath), thereby crosslinking the edible membrane or second edible membrane to provide an edible bioreactor.

35-43. (canceled)
